SEQUENCE LISTING (76 sequences)

SEQ ID NO: 1 - protein, 21 aa, Artificial Sequence
  source: /note=“Description of Artificial Sequence Synthetic peptide”
  MOD_RES (1)..(1): Any hydrophobic residue
  MOD_RES (2)..(2): Any amino acid
  MOD_RES (3)..(3): Any hydrophobic residue
  MOD_RES (5)..(5): Any amino acid
  MOD_RES (6)..(6): Any amino acid
  MOD_RES (7)..(7): Any hydrophobic residue
  MOD_RES (8)..(8): Any amino acid
  MOD_RES (9)..(9): Any hydrophobic residue
  MOD_RES (10)..(10): Any amino acid
  MOD_RES (14)..(14): Any amino acid
  MOD_RES (15)..(15): Any amino acid
  MOD_RES (17)..(17): Any hydrophobic residue
  MOD_RES (18)..(18): Any amino acid
  MOD_RES (19)..(19): Any hydrophobic residue
  MOD_RES (20)..(20): Any amino acid
XXXGXXXXXXCIIXXGXXXXG

SEQ ID NO: 2 - protein, 300 aa, Artificial Sequence
  source: /note=“Description of Artificial Sequence Synthetic polypeptide”
MGASSPRSPEPVGPPAPGLPFCCGGSLLAVVVLLALPVAWGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGNTVIWDNETPICDRIPCGLPPTITNGDFISTNRENFHYGSVVTYRCNPGSGGRKVFELVGEPSIYCTSNDDQVGIWSGPAPQCIIPNSGGGSGGGTPEGCEQVLTGKRLMQCLPNPEDVKMALEVYKLSLEIEQLELQRDSARQSTLDKEL

SEQ ID NO: 3 - protein, 300 aa, Artificial Sequence
  source: /note=“Description of Artificial Sequence Synthetic polypeptide”
MGASSPRSPEPVGPPAPGLPFCCGGSLLAVVVLLALPVAWGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNSGGGSGGGTPEGCEQVLTGKRLMQCLPNPEDVKMALEVYKLSLEIEQLELQRDSARQSTLDKEL

SEQ ID NO: 4 - protein, 493 aa, Artificial Sequence
  source: /note=“Description of Artificial Sequence Synthetic polypeptide”
MWWRLWWLLLLLLLLWPMVWAAATPEGCEQVLTGKRLMQCLPNPEDVKMALEVYKLSLEIEQLELQRDSARQSTLDKELGGGSKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSAGSGKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPDGSAKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 5 - protein, 206 aa, Artificial Sequence
  source: /note=“Description of Artificial Sequence Synthetic polypeptide”
MWWRLWWLLLLLLLLWPMVWAAAGRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKSGGGSGGGTPEGCEQVLTGKRLMQCLPNPEDVKMALEVYKLSLEIEQLELQRDSARQSTLDKEL

SEQ ID NO: 6 - protein, 200 aa, Artificial Sequence
  source: /note=“Description of Artificial Sequence Synthetic polypeptide”
MWWRLWWLLLLLLLLWPMVWAAATPEGCEQVLTGKRLMQCLPNPEDVKMALEVYKLSLEIEQLELQRDSARQSTLDKELGGGSSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILE

SEQ ID NO: 7 - protein, 416 aa, Artificial Sequence
  source: /note=“Description of Artificial Sequence Synthetic polypeptide”
MWWRLWWLLLLLLLLWPMVWAAAGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNSGGGGSGGGGSGGGGSTPEGCEQVLTGKRLMQCLPNPEDVKMALEVYKLSLEIEQLELQRDSARQSTLDKELSGGGSGGGSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILE

SEQ ID NO: 8 - protein, 570 aa, Artificial Sequence
  source: /note=“Description of Artificial Sequence Synthetic polypeptide”
MWWRLWWLLLLLLLLWPMVWAAAGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNSGGGGSGGGGSGGGGSDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRSGGGGSGGGGSGGGGSGQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGNTVIWDNETPICDRIPCGLPPTITNGDFISTNRENFHYGSVVTYRCNPGSGGRKVFELVGEPSIYCTSNDDQVGIWSGPAPQCIIPN

SEQ ID NO: 9 - protein, 194 aa, Artificial Sequence
  source: /note=“Description of Artificial Sequence Synthetic polypeptide”
GQCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCII

SEQ ID NO: 10 - protein, 194 aa, Artificial Sequence
  source: /note=“Description of Artificial Sequence Synthetic polypeptide”
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCII

SEQ ID NO: 11 - protein, 194 aa, Artificial Sequence
  source: /note=“Description of Artificial Sequence Synthetic polypeptide”
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 12 - protein, 128 aa, Artificial Sequence
  source: /note=“Description of Artificial Sequence Synthetic polypeptide”
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVS

SEQ ID NO: 13 - protein, 304 aa, Artificial Sequence
  source: /note=“Description of Artificial Sequence Synthetic polypeptide”
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTL

SEQ ID NO: 14 - protein, 178 aa, Artificial Sequence
  source: /note=“Description of Artificial Sequence Synthetic polypeptide”
STCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVE

SEQ ID NO: 15 - protein, 84 aa, Artificial Sequence
  source: /note=“Description of Artificial Sequence Synthetic polypeptide”
SCVNPPTVQNAHILSRQMSKYPSGERVRYECRSPYEMFGDEEVMCLNGNWTEPPQCKDSTGKCGPPPPIDNGDITSFPLSVYAP

SEQ ID NO: 16 - protein, 126 aa, Artificial Sequence
  source: /note=“Description of Artificial Sequence Synthetic polypeptide”
TISAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCE

SEQ ID NO: 17 - protein, 122 aa, Artificial Sequence
  source: /note=“Description of Artificial Sequence Synthetic polypeptide”
KCGPPPPISNGDTTSFLLKVYVPQSRVEYQCQPYYELQGSNYVTCSNGEWSEPPRCIHPCIITEENMNKNNIKLKGRSDRKYYAKTGDTIEFMCKLGYNANTSILSFQAVCREGIVEYPRCE

SEQ ID NO: 18 - protein, 123 aa, Artificial Sequence
  source: /note=“Description of Artificial Sequence Synthetic polypeptide”
EKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 19 - protein, 424 aa, Artificial Sequence
  source: /note=“Description of Artificial Sequence Synthetic polypeptide”
ECHVPILEANVDAQPKKESYKVGDVLKFSCRKNLIRVGSDSVQCYQFGWSPNFPTCKGQVRSCGPPPQLSNGEVKEIRKEEYGHNEVVEYDCNPNFIINGPKKIQCVDGEWTTLPTCVEQVKTCGYIPELEYGYVQPSVPPYQHGVSVEVNCRNEYAMIGNNMITCINGIWTELPMCVATHQLKRCKIAGVNIKTLLKLSGKEFNHNSRIRYRCSDIFRYRHSVCINGKWNPEVDCTEKREQFCPPPPQIPNAQNMTTTVNYQDGEKVAVLCKENYLLPEAKEIVCKDGRWQSLPRCVESTAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 20 - protein, 250 aa, Artificial Sequence
  source: /note=“Description of Artificial Sequence Synthetic polypeptide”
CGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRG

SEQ ID NO: 21 - protein, 250 aa, Artificial Sequence
  source: /note=“Description of Artificial Sequence Synthetic polypeptide”
EEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLK

SEQ ID NO: 22 - protein, 238 aa, Artificial Sequence
  source: /note=“Description of Artificial Sequence Synthetic polypeptide”
NCGPPPTLSFAAPMDITLTETRFKTGTTLKYTCLPGYVRSHSTQTLTCNSDGEWVYNTFCIYKRCRHPGELRNGQVEIKTDLSFGSQIEFSCSEGFFLIGSTTSRCEVQDRGVGWSHPLPQCEIVKCKPPPDIRNGRHSGEENFYAYGFSVTYSCDPRFSLLGHASISCTVENETIGVWRPSPPTCEKITCRKPDVSHGEMVSGFGPIYNYKDTIVFKCQKGFVLRGSSVIHCDADSK

SEQ ID NO: 23 - protein, 245 aa, Artificial Sequence
  source: /note=“Description of Artificial Sequence Synthetic polypeptide”
SCCTIPSRPINMKFKNSVETDANANYNIGDTIEYLCLPGYRKQKMGPIYAKCTGTGWTLFNQCIKRRCPSPRDIDNGHLDIGGVDFGSSITYSCNSGYYLIGEYKSYCELGSTGSMVWNPKAPICESVKCQLPPSISNGRHNGYNDFYTDGSVVTYSCNSGYSLIGNSGVLCSGGEWSNPPTCQIVKCPHPTILNGYLSSGFKRSYSYNDNVDFTCKYGYKLSGSSSSTCSPGNTWQPELPKCVR

SEQ ID NO: 24 - protein, 244 aa, Artificial Sequence
  source: /note=“Description of Artificial Sequence Synthetic polypeptide”
CCTIPSRPINMKFKNSVETDANANYNIGDTIEYLCLPGYRKQKMGPIYAKCTGTGWTLFNQCIKRRCPSPRDIDNGQLDIGGVDFGSSITYSCNSGYHLIGESKSYCELGSTGSMVWNPEAPICESVKCQSPPSISNGRHNGYEDFYTDGSVVTYSCNSGYSLIGNSGVLCSGGEWSDPPTCQIVKCPHPTISNGYLSSGFKRSYSYNDNVDFKCKYGYKLSGSSSSTCSPGNTWKPELPKCVR

SEQ ID NO: 25 - protein, 58 aa, Artificial Sequence
  source: /note=“Description of Artificial Sequence Synthetic polypeptide”
WETPEGCEQVLTGKRLMQCLPNPEDVKMALEVYKLSLEIEQLELQRDSARQSTLDKEL

SEQ ID NO: 26 - protein, 55 aa, Artificial Sequence
  source: /note=“Description of Artificial Sequence Synthetic polypeptide”
PKPECEKALLAFQESKNLCEAMENFMQQLKESGMTMEELKYSLELKKAELKAKLL

SEQ ID NO: 27 - protein, 119 aa, Artificial Sequence
  source: /note=“Description of Artificial Sequence Synthetic polypeptide”
GRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQK

SEQ ID NO: 28 - protein, 259 aa, Artificial Sequence
  source: /note=“Description of Artificial Sequence Synthetic polypeptide”
AAQPADIVLTQTPSSLPVSVGEKVTMTCKSSQTLLYSNNQKNYLAWYQQKPGQSPKLLISWAFTRKSGVPDRFTGSGSGTDFTLTIGSVKAEDLAVYYCQQYSNYPWTFGGGTRLEIKRGGGGSGGGGSGGGGSGGGGSEVQLQQSGPEVVKTGASVKISCKASGYSFTGYFINWVKKNSGKSPEWIGHISSSYATSTYNQKFKNKAAFTVDTSSSTAFMQLNSLTSEDSAVYYCVRSGNYEEYAMDYWGQGTSVTVSS

SEQ ID NO: 29 - protein, 282 aa, Artificial Sequence
  source: /note=“Description of Artificial Sequence Synthetic polypeptide”
ETVIMKYLLPTAAAGLLLLAAQPAMAQVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSGGGGSGGGGSGGGGSENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAAAEQKLISEEDLN

SEQ ID NO: 30 - protein, 234 aa, Artificial Sequence
  source: /note=“Description of Artificial Sequence Synthetic polypeptide”
EVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSGGSGGDIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 31 - protein, 245 aa, Artificial Sequence
  source: /note=“Description of Artificial Sequence Synthetic polypeptide”
METITVSTPIKQIFPDDAFAETIKANLKKKSVTDAVTQNELNSIDQIIANNSDIKSVQGIQYLPNVRYLALGGNKLHDISALKELTNLTYLMLHYNQLQILPNGVFDKLTNLKELYLSENQLQSLPDGVFDKLTNLTELDLARNQLQSLPKGVFDKLTQLKDLRLYQNQLKSVPDGVFDRLTSLQYIWLHDNPWDCTCPGIRYLSEWINKHSGVVRNSAGSVAPDSAKCSGSGKPVRSIICPTLE

SEQ ID NO: 32 - protein, 138 aa, Artificial Sequence
  source: /note=“Description of Artificial Sequence Synthetic polypeptide”
TGQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSSGSMDPGGS

SEQ ID NO: 33 - protein, 127 aa, Artificial Sequence
  source: /note=“Description of Artificial Sequence Synthetic polypeptide”
GQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVT

SEQ ID NO: 34 - protein, 126 aa, Artificial Sequence
  source: /note=“Description of Artificial Sequence Synthetic polypeptide”
GQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTAL

SEQ ID NO: 35 - protein, 67 aa, Artificial Sequence
  source: /note=“Description of Artificial Sequence Synthetic polypeptide”
LSCCTIPSRPINMKFKNSVETDANANYNIGDTIEYLCLPGYRKQKMGPIYAKCTGTGWTLFNQCIKR

SEQ ID NO: 36 - protein, 66 aa, Artificial Sequence
  source: /note=“Description of Artificial Sequence Synthetic polypeptide”
SDACEEPPTFEAMELIGKPKPYYEIGERVDYKCKKGYFYIPPLATHTICDRNHTWLPVSDDACYRE

SEQ ID NO: 37 - protein, 63 aa, Artificial Sequence
  source: /note=“Description of Artificial Sequence Synthetic polypeptide”
LGHCQAPDHFLFAKLKTQTNASDFPIGTSLKYECRPEYYGRPFSITCLDNLVWSSPKDVCKRK

SEQ ID NO: 38 - protein, 63 aa, Artificial Sequence
  source: /note=“Description of Artificial Sequence Synthetic polypeptide”
WGQCNAPEWLPFARPTNLTDEFEFPIGTYLNYECRPGYSGRPFSIICLKNSVWTGAKDRCRRK

SEQ ID NO: 39 - protein, 65 aa, Artificial Sequence
  source: /note=“Description of Artificial Sequence Synthetic polypeptide”
LGNCGPPPTLSFAAPMDITLTETRFKTGTTLKYTCLPGYVRSHSTQTLTCNSDGEWVYNTFCIYK

SEQ ID NO: 40 - protein, 63 aa, Artificial Sequence
  source: /note=“Description of Artificial Sequence Synthetic polypeptide”
DCGLPPDVPNAQPALEGRTSFPEDTVITYKCEESFVKIPGEKDSVICLKGSQWSDIEEFCNRS

SEQ ID NO: 41 - protein, 63 aa, Artificial Sequence
  source: /note=“Description of Artificial Sequence Synthetic polypeptide”
GRTCPKPDDLPFSTVVPLKTFYEPGEEITYSCKPGYVSRGGMRKFICPLTGLWPINTLKCTPR

SEQ ID NO: 42 - protein, 66 aa, Artificial Sequence
  source: /note=“Description of Artificial Sequence Synthetic polypeptide”
AEDCNELPPRRNTEILTGSWSDQTYPEGTQAIYKCRPGYRSLGNVIMVCRKGEWVALNPLRKCQKR

SEQ ID NO: 43 - protein, 67 aa, Artificial Sequence; identical to SEQ ID NO: 35
  source: /note=“Description of Artificial Sequence Synthetic polypeptide”

SEQ ID NO: 44 - protein, 64 aa, Artificial Sequence
  source: /note=“Description of Artificial Sequence Synthetic polypeptide”
KRRCPSPRDIDNGQLDIGGVDFGSSITYSCNSGYHLIGESKSYCELGSTGSMVWNPEAPICESV

SEQ ID NO: 45 - protein, 65 aa, Artificial Sequence
  source: /note=“Description of Artificial Sequence Synthetic polypeptide”
RETCPYIRDPLNGQAVPANGTYEFGYQMHFICNEGYYLIGEEILYCELKGSVAIWSGKPPICEKV

SEQ ID NO: 46 - protein, 64 aa, Artificial Sequence
  source: /note=“Description of Artificial Sequence Synthetic polypeptide”
RKSCKTPPDPVNGMVHVITDIQVGSRINYSCTTGHRLIGHSSAECILSGNAAHWSTKPPICQRI

SEQ ID NO: 47 - protein, 64 aa, Artificial Sequence
  source: /note=“Description of Artificial Sequence Synthetic polypeptide”
RKSCRNPPDPVNGMVHVIKGIQFGSQIKYSCTKGYRLIGSSSATCIISGDTVIWDNETPICDRI

SEQ ID NO: 48 - protein, 64 aa, Artificial Sequence
  source: /note=“Description of Artificial Sequence Synthetic polypeptide”
YKRCRHPGELRNGQVEIKTDLSFGSQIEFSCSEGFFLIGSTTSRCEVQDRGVGWSHPLPQCEIV

SEQ ID NO: 49 - protein, 67 aa, Artificial Sequence
  source: /note=“Description of Artificial Sequence Synthetic polypeptide”
NRSCEVPTRLNSASLKQPYITQNYFPVGTVVEYECRPGYRREPSLSPKLTCLQNLKWSTAVEFCKKK

SEQ ID NO: 50 - protein, 60 aa, Artificial Sequence
  source: /note=“Description of Artificial Sequence Synthetic polypeptide”
PRVCPFAGILENGAVRYTTFEYPNTISFSCNTGFYLNGADSAKCTEEGKWSPELPVCAPI

SEQ ID NO: 51 - protein, 60 aa, Artificial Sequence
  source: /note=“Description of Artificial Sequence Synthetic polypeptide”
ESTCGDIPELEHGWAQLSSPPYYYGDSVEFNCSESFTMIGHRSITCIHGVWTQLPQCVAI

SEQ ID NO: 52 - protein, 63 aa, Artificial Sequence
  source: /note=“Description of Artificial Sequence Synthetic polypeptide”
KRRCPSPRDIDNGQLDIGGVDFGSSITYSCNSGYHLIGESKSYELGSTGSMVWNPEAPICESV

SEQ ID NO: 53 - protein, 60 aa, Artificial Sequence
  source: /note=“Description of Artificial Sequence Synthetic polypeptide”
SVKCQSPPSISNGRHNGYEDFYTDGSVVTYSCNSGYSLIGNSGVLCSGGEWSDPPTCQIV

SEQ ID NO: 54 - protein, 68 aa, Artificial Sequence
  source: /note=“Description of Artificial Sequence Synthetic polypeptide”
KVLCTPPPKIKNGKHTFSEVEVFEYLDAVTYSCDPAPGPDPFSLIGESTIYCGDNSVWSRAAPECKVV

SEQ ID NO: 55 - protein, 73 aa, Artificial Sequence
  source: /note=“Description of Artificial Sequence Synthetic polypeptide”
RIPCGLPPTIANGDFISTNRENFHYGSVVTYRCNPGSGGRKVFELVGEPSIYCTSNDDQVGIWSGPAPQCIIP

SEQ ID NO: 56 - protein, 73 aa, Artificial Sequence
  source: /note=“Description of Artificial Sequence Synthetic polypeptide”
RIPCGLPPTITNGDFISTNRENFHYGSVVTYRCNPGSGGRKVFELVGEPSIYCTSNDDQVGIWSGPAPQCIIP

SEQ ID NO: 57 - protein, 66 aa, Artificial Sequence
  source: /note=“Description of Artificial Sequence Synthetic polypeptide”
IVKCKPPPDIRNGRHSGEENFYAYGFSVTYSCDPRFSLLGHASISCTVENETIGVWRPSPPTCEKI

SEQ ID NO: 58 - protein, 64 aa, Artificial Sequence
  source: /note=“Description of Artificial Sequence Synthetic polypeptide”
KKSCPNPGEIRNGQIDVPGGILFGATITFSCNTGYKLFGSTSSFCLISGSSVQWSDPLPECREI

SEQ ID NO: 59 - protein, 65 aa, Artificial Sequence
  source: /note=“Description of Artificial Sequence Synthetic polypeptide”
PIICPPPSIPTFATLRVYKPSAGNNSLYRDTAVFECLPQHAMFGNDTITCTTHGNWTKLPECREV

SEQ ID NO: 60 - protein, 66 aa, Artificial Sequence
  source: /note=“Description of Artificial Sequence Synthetic polypeptide”
VVKCLPVTAPENGKIVSSAMEPDREYHFGQAVRFVCNSGYKIEGDEEMHCSDDGFWSKELPKCVEI

SEQ ID NO: 61 - protein, 61 aa, Artificial Sequence
  source: /note=“Description of Artificial Sequence Synthetic polypeptide”
SVKCQSPPSISNGRHNGYEDFYTDGSVVTYSCBSGYSLIGNSGVLCSGGEEWSDPPTCQIV

SEQ ID NO: 62 - protein, 61 aa, Artificial Sequence
  source: /note=“Description of Artificial Sequence Synthetic polypeptide”
IVKCPHPTISNGYLSSGFKRSYSYNDNVDFKCKYGYKLSGSSSSTCSPGNTWKPELPKCVR

SEQ ID NO: 63 - protein, 62 aa, Artificial Sequence
  source: /note=“Description of Artificial Sequence Synthetic polypeptide”
VVKCRFPVVENGKQISGFGKKFYYKATVMFECDKGFYLDGSDTIVCDSNSTWDPPVPKCLKG

SEQ ID NO: 64 - protein, 62 aa, Artificial Sequence
  source: /note=“Description of Artificial Sequence Synthetic polypeptide”
PNKCTPPNVENGILVSDNRSLFSLNEVVEFRCQPGFVMKGPRRVKCQALNKWEPELPSCSRV

SEQ ID NO: 65 - protein, 62 aa, Artificial Sequence; identical to SEQ ID NO: 64
  source: /note=“Description of Artificial Sequence Synthetic polypeptide”

SEQ ID NO: 66 - protein, 62 aa, Artificial Sequence
  source: /note=“Description of Artificial Sequence Synthetic polypeptide”
KITCRKPDVSHGEMVSGFGPIYNYKDTIVFKCQKGFVLRGSSVIHCDADSKWNPSPPACEPN

SEQ ID NO: 67 - protein, 65 aa, Artificial Sequence
  source: /note=“Description of Artificial Sequence Synthetic polypeptide”
EIYCPAPPQIDNGIIQGERDHYGYRQSVTYACNKGFTMIGEHSIYCTVNNDEGEWSGPPPECRGK

SEQ ID NO: 68 - protein, 62 aa, Artificial Sequence
  source: /note=“Description of Artificial Sequence Synthetic polypeptide”
EVKCPFPSRPDNGFVNYPAKPTLYYKDKATFGCHDGYSLDGPEEIECTKLGNWSAMPSCKAS

SEQ ID NO: 69 - protein, 59 aa, Artificial Sequence
  source: /note=“Description of Artificial Sequence Synthetic polypeptide”
EISCKSPDVINGSPISQKIIYKENERFQYKCNMGYEYSERGDAVCTESGWRPLPSCEEK

SEQ ID NO: 70 - protein, 61 aa, Artificial Sequence; identical to SEQ ID NO: 62
  source: /note=“Description of Artificial Sequence Synthetic polypeptide”

SEQ ID NO: 71 - protein, 137 aa, Rattus norvegicus
AYMYHSAFSVGLETRVTVPNVPIRFTKIFYNQQNHYDGSTGKFHCNIPGLYYFSYHITVYMKDVKVSLFKKDKAVLFTYDQYQEKNVDQASGSMLLHLEVGDQVWLQVYGEGDNNGLYADNVNDSTFTGFLLYHDTN

SEQ ID NO: 72 - protein, 139 aa, Mus musculus
GATQKVAFSALRTINSPLRPNQVIRFEKVITNANENYEPRNGKFTCKVPGLYYFTYHASSRGNLCVNLVRGRDRDSMQKVVTFCDYAQNTFQVTTGGVVLKLEQEEVVHLQATDKNSLLGIEGANSIFTGFLLFPDMDA

SEQ ID NO: 73 - protein, 152 aa, Homo sapiens
EEFPRDLVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 74 - protein, 146 aa, Homo sapiens
TLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 75 - protein, 146 aa, Homo sapiens
GDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 76 - protein, 123 aa, Artificial Sequence
  source: /note=“Description of Artificial Sequence Synthetic polypeptide”
KCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAK

(19) United States
(12) Patent Application Publication
(10) Pub. No.: US 2022/0220169 A1
(43) Pub. Date: Jul. 14, 2022

(54) Modular Therapeutics for the Treatment of Inflammatory Diseases and Cancer

(71) Applicant: INSIDEOUTBIO, INC., Charlestown, MA (US)

(72) Inventor: Alan Gordon Herbert, Charlestown, MA (US)

(21) Appl. No.: 17/595,378

(22) PCT Filed: May 15, 2020
(86) PCT No.: PCT/US2020/033022

§ 371 Date: Nov. 15, 2021

Related U.S. Application Data
(60) Provisional application No. 62/848,345, filed on May 15, 2019.

(51) Int. Cl. C07K 14/47; C07K 14/705

(52) U.S. Cl. CPC C07K 14/472; A61K 38/00; C07K 14/705

(57) ABSTRACT

Compositions and methods to regulate immune responses by fusing Complement Control Protein domains and extra-cellular Complement Receptor domains to a scaffold through flexible linkers for treatment of immunological diseases and disorders oar described.

### RELATED APPLICATIONS

This application claims the benefit under 35 U.S.C. § 119(e) of U.S. Provisional Application No. 62/848,345, filed on May 15, 2019, which is incorporated herein by reference in its entirety.

### BACKGROUND OF THE INVENTION

The game of life requires that organisms attack, accommodate or dismiss threats posed by other players. At the same time, they must prepare for a possible rematch. First, they must identify the threat. Initially a simple system arose for labeling self differently from non-self that preceded innovations based on gene rearrangements that underlie the adaptive immune system [1]. Referred to as innate immunity, the system used complement to label host and non-host with different proteolytic fragments derived from complement component C3 and C4, enabling targeted responses against an invader. Initially the system worked intracellularly to protect unicellular organisms against pathogens. Then as multi-cellular creatures emerged, complement evolved new protections, first at the membrane, then in the space between cells that enabled killing, phagocytosis or neglect of other players according to the attached label. The protections also extended to the removal of dead host cells and the elimination of abnormal ones. FIG. 1 is a schematic showing the complement pathways.

The modern complement system uses multiples activators and regulators to identify threats both from invaders and from self. Many of these elements arose from an ancient set of building blocks that were duplicated and adapted to make new enzymes, activators, regulators and receptors [2]. The common ancestor of eumetazoa used a system based only on complement component 3 (C3) convertase, factor B (FB) protease and the mannan binding lectin serine peptidase (MASP) activator [3]. The system's earliest regulators, dating back to teleost fish, were membrane cofactor protein (MCP) and Factor I (FI) [4]. In the original schema, MASP, activated by non-self carbohydrates, cleaved FB leading to the proteolytic activation of the C3 Convertase, which was subsequently inactivated by FI bound to MCP. From this start evolved the classical pathway of complement activation initiated by antigen-bound antibody and the “always on” intravascular alternative pathway for labelling invaders that lack the ability to deactivate the cascade. The alternative pathway is self-amplifying and magnifies the response initiated by the other C3 convertases.

Regulators of complement activation (RCA) arose through the duplication and modification of the MCP complement control protein modules (CCP), [5].

The focus of many therapeutic approaches to treat a variety of diseases associated with these complement pathways, including any hyper-proliferative cellular diseases such as cancer, inflammatory diseases, auto-inflammatory diseases and transplant rejection comprise targeting the regulatory components of the complement pathways. However, there still remains a need for compositions and methods with specific targeted activation or inhibition of complement activity to treat these diseases.

### SUMMARY OF THE INVENTION

The present invention encompasses compositions, method of making the constructs described herein, and methods of using those compositions for the targeted activation or inhibition of immune cells to stimulate a cellular or humoral immune response against tumors or pathogens in a host/subject. The invention specifically comprises methods to regulate the immune response by activating Complement Control Proteins (specifically, actCCPs) on the surface of an antigen-bearing cell using CCPs, be that a tumor cell or a dendritic cell or another antigen presenting cell or an extracellular vesicle, where the CCPs are directed to the site of action by a targeting ligand that may be one, or more, CCP, or another peptide or reagent. As described herein, the methods of the present invention will induce an immune response against tumor specific proteins that makes all parts of the tumor susceptible to attack by the immune system both at the site of administration as well as at more distant sites throughout the body. As also described, methods using a different set of CCPs or complement receptor extracellular domains (inhCCP) will allow inhibition of immune responses associated with inflammatory diseases like rheumatoid arthritis or autoimmune diseases such as systemic lupus erythematosus, multiple sclerosis, glomerulonephritis or due to allergies or arising from tissue transplantation between individuals.

The present invention encompasses constructs/compositions/therapeutic compositions comprising one, or more CCPs (either activating or inhibitory), one, or more targeting domains (TDs) linked to a scaffold. (see for example FIGS. 4 and 5). The fusion constructs are encoded by DNA that contains all the elements for the production of a actCCPs or inhCCPs in the appropriate host cell or host tissue containing suitable cells, and delivered to the host either as a nucleic acid construct in an expression vector, as an RNA, or as a protein by either local or systemic delivery. The constructs can comprise multimers of one, or more CCP domains typically numbering 3-4 protein domains/modules, but can comprise more than 3-4 or fewer than 3-4 domains, as long as configuration of the expressed constructs does not restrict or imped the biological activity of the expressed construct. The CCP domains can be all the same CCP or different where one, or more of the CCP domains are from different CCPs. More specifically, a construct can be composed of about 3-4 complement control protein modules (CCP) with specificity for other components of the complement system and designed to alter the assembly of complement convertases, cause decay of such convertases or change the complement proteolytic products that they produce. Another set of constructs uses the extracellular domains of complement receptors. Therapeutic compositions described herein incorporate ligands to target particular surfaces or receptors.

More specifically the constructs of the present invention comprise a scaffold component, typically a protein, to which the CCP is linked by a flexible linker of suitable length. The length of the linker can be determined by one skilled in the art such that the length is sufficient to link the CCPs to the scaffold without sterically interfering with the activity of the CCP or targeting domains. The linker can be a protein or a chemical linker such as those well-known to those of skill in the art. The CCP(s) can be attached to the scaffold via the N-amino acid or C-carboxy termini of the CCP(s). The use of scaffolding is an important feature of the present invention. The scaffolds allow incorporation of more than one copy of an actCCP or inhCCP in the construct/composition while greatly diminishing recombination of nucleic acid encoding them that would otherwise lead to undesired products during their production in host cells.

The scaffolds allow tuning of the apparent affinity of the construct/composition by varying the scaffold to change the number of monomers in the final assembly. The scaffold protein itself can act as a targeting domain by binding a cognate receptor, or provide a framework that allows incorporation of targeting ligands, either by fusing the ligand to the same construct as an actCCP or inhCCP to produce a homomeric construct or by fusing the ligand with one of the scaffold monomers and fusing the actCCP or inhCCP to another to produce a heteromeric construct (see for example FIGS. 6-9). This invention also includes the use of multimeric scaffolds to which only the targeting ligands are directly attached. This approach allows the use of low affinity ligands that are part of, or attached to a multimeric scaffold, favoring interactions with surfaces where the number of receptors is high in disease states but low or absent in normal cells.

The present invention further comprises constructs produced by linking (also referred to herein as fusing) actCCPs or inhCCPs to scaffolds composed of a single unit to produce therapeutics with actCCPs or inhCCPs at both ends of the scaffold or attached to them through a chemical modification (FIG. 7 to FIG. 9). These include scaffolds that have a high affinity for a cell surface receptor, or to which targeting ligands are attached. This approach allows the use of high affinity ligands to target surfaces where the number of receptors is high in disease states but low or absent in normal cells.

The constructs of the present invention have biological/therapeutic activity and can activate or inhibit complement pathways according to the components that make up the construct. For example, as described herein, the present invention comprises methods to inactivate complement convertases of different complement activation pathways using the actCCPs or inhCCPs listed in FIG. 10, or CCPs comprising up to about 85%, 90%, 95%, 96%, 97%, 98% or 99% homology or sequence identity to the CCPs described herein, each varying by their ability to inhibit convertase activation, their DAA (decay-accelerating activity) and CA (cofactor activity) and the complement pathways they act upon. These components enable the construction of both actCCP and inhCCP therapeutics using the scaffolds and targeting ligands named.

The constructs are designed for delivery either as nucleic acid therapeutics, or as manufactured proteins administered either locally or systemically for the treatment of disease.

The purpose of therapeutic compositions with inhCCPs is to inhibit immune responses against antigens on the same surface as the therapeutic binds. The fusion constructs may prevent convertase activation, accelerate decay of the convertase or direct Factor I to increase the density of iC3b on the cell surface.

The purpose of therapeutic compositions with actCCPs that have cofactor activity for the generation of C3d and/or C4d on the cell surface is to stimulate immune responses against antigens on the same surface as the therapeutic binds by increasing formation of C3d and/or C4d on or by attaching C3d and/or C4d to the surface targeted.

Methods described herein include the construction of a fusion protein containing actCCPs or inhCCPs fused to a C4BP scaffold (FIG. 6). Methods described herein include the construction of a fusion protein containing actCCPs or inhCCPs fused to C4BP scaffold and to ligands targeting it to specific surfaces. Natural ligands or their variants that bind immunoregulatory receptors including PD-1 and the globular domain of members of the C1qTNF family are given as examples [6, 7] (FIGS. 7 and 8).

Methods described herein include the construction of a fusion protein containing actCCPs or inhCCPs sequence fused directly to a PD1 scaffold or to variants with enhanced affinity for its receptors (FIG. 7).

Methods described herein include the construction of a fusion protein containing ligands directly fused to a single chain Clq/TNF globular domain, either wildtype or mutant, that targets it to a surface bearing cognate receptors (FIG. 8).

Methods described herein include the construction of a fusion protein containing actCCPs or inhCCPs fused to an antigen-binding scaffold. Examples include antigen-binding scaffolds that target Tumor Necrosis Factor Receptors, HER2/NEU (ERBB2) Vascular Endothelial Growth Factor Receptors (KDR, FLT1, FLT4) and Epithelial Growth Factor Receptors (EGFR, ERBB3, ERBB4) are described (FIG. 9).

Methods described herein include the construction of a fusion protein with a single chain C1q globular domain fused to the C4BP scaffold for inhibiting the binding of C1q to surfaces that would otherwise activate complement or promote non-inflammatory phagocytosis of cancer cells (FIG. 6).

Methods described herein include the construction of a fusion protein with a single chain PD-1 domain fused to the C4BP scaffold to inhibit binding of PD-1 bearing cells to surfaces with a PD-1 receptor that would otherwise inhibit their function (FIG. 6).

In one embodiment, the fusion protein is expressed from a polynucleotide composed of either DNA or RNA introduced to the target cell and that contains sequences necessary for the cellular machinery to produce, assemble and export it to the cell surface membrane. The expression of the fusion protein may be limited to a particular cell type by use of appropriate promoters and enhancers known to one skilled in the art. The agent may be delivered to the target cell using known delivery vehicles, including without limitation, viral vectors, nanoparticles, liposomes or exosomes that may or may not contained ligands for the target cell on their surface. If the delivery of the construct is via a viral vector, the viral vector can comprise any suitable replicating or non-replicating viral vector for targeting and delivery of the construct into a cell and can be for example, adenovirus, adeno-associated virus or lentivirus. Alternatively, local delivery by injection, electroporation or other mechanical or electrophysiological mechanisms can be used to target a specific tissue or disease location.

Another embodiment of the present invention is the delivery of a premade protein to the surface of the target cell using known delivery vehicles, including without limitation, viral vectors, nanoparticles, liposomes, transfectants, transductants or exosomes that may or may not contained ligands for the target cell on their surface.

The methods of the present invention comprise the use of an expression vector that targets a cell, wherein the vector comprises a nucleic acid construct that expresses actCCPs or inhCCPs plus scaffold with or without an additional active, wildtype, or mutant, targeting ligand, or an expression vector encoding a protein that activates expression in the target cell of actCCPs or inhCCPs plus scaffold with or without an active, wildtype, or mutant, targeting ligand. As a result of contacting a target cell, the immunogenicity of the cell is enhanced by an activating therapeutic and the tumor cell becomes more susceptible to attack by the immune system. On the other hand, when inhibitory therapeutics are delivered to a cell, the stimulation of negative regulatory immune cells is enhanced, leading to a suppression or inhibition of immune responses.

The methods of the present invention include actCCPs or inhCCPs with a mutation that create a novel DAA or CA specificity as exemplified by the D109N mutation in CR1 site 1 constructs [8].

The present invention also covers the delivery of a defined antigen to the target cell along with the actCCPs. In the case of actCCPs, codelivery with a defined antigen increases immune response to that antigen so as to constitute a vaccine against tumors or pathogens that bear the specified antigen. The defined antigen may be delivered in a number of ways as known to those experienced in the art but not involving fusion with the actCCP.

The present invention also covers the delivery of a defined antigen to the target cell along with the inhCCPs. In the case of inhCCPs, co-delivery with a defined antigen decreases immune response to that antigen so as to constitute a vaccine against allergens or other antigens causing activation of the immune system leading to the disease associated with that antigen. The co-delivery with a defined antigen decreases or suppresses immune responses associated with allergy, inflammation, autoimmunity and transplantation triggered by the specified antigen. The defined antigen may be delivered in a number of ways as known to those experienced in the art but is not involving fusion with the inhCCP.

The method of the present invention describes delivery of a defined antigen with a scaffold that localizes the agent to the cell surface along with actCCPs or inhCCPs to induce an immune response against the antigen in the case of actCCPs or to inhibit or suppress it in the case of inhCCPs.

In a particular embodiment, the subject in the methods of this invention is a mammal, and more particularly, the mammal is a human and can activate immunity using actCCPs or inhibit it using inhCCPs.

A particular embodiment of the present invention encompasses methods of treating cancer in a mammal (e.g., a human patient or individual) using actCCP, preventing metastasis of the cancer and protecting against reoccurrence of the cancer wherein administering to the individual a therapeutically effective amount of the agent increases the expression of actCCPs in and on the tumor cells or in the tumor micro-environment.

Another embodiment of the present invention is to create a vaccine against tumors that express a defined antigen so as to provoke an immune response to protect an individual against that tumor type, including applications where the vaccine is delivered locally, to lymph nodes, to other tissues or systemically by injection.

Another embodiment of the present invention using actCCP is to create a vaccine against a pathogen that expresses a defined antigen so as to provoke an immune response to protect an individual against that pathogen.

The methods described herein using actCCP can be used to treat many different forms of cancers. For example, the cancer can be ovarian, breast, colon or lung cancer. The method of treating cancer can further encompass administering the actCCP agents concurrently with, or sequentially before or after, or in conjunction with, at least one, or more additional or complementary cancer treatments suitable for the treatment of the specific cancer. For example, without limitation, the complementary cancer treatment can be selected from a therapy comprising checkpoint inhibitor; a proteasome inhibitor; immunotherapeutic agent; radiation therapy or chemotherapy. Other suitable additional or complementary cancer therapies are known to those of skill in the art.

A particular embodiment of the present invention encompasses methods of treating inflammatory and autoimmune diseases in an individual, associated with complement activation wherein administering to the individual of a therapeutically effective amount of an agent increases the expression of inhCCPs at surfaces with the antigen, leading to inhibition of complement convertases, increased DAA and CA, increased iC3b production with inhibition of C3d production.

The method of treating complement-mediated inflammatory disease can further encompass administering the inhCCP agents concurrently with, or sequentially before or after, or in conjunction with, at least one, or more additional or complementary anti-inflammatory treatments suitable for the treatment of the specific disease. For example, without limitation, the inflammatory disease treatment can be selected from a therapy comprising steroids; an anti-proliferative agent; a proteasome inhibitor; immunosuppressive agent or radiation therapy. Other suitable additional or complementary disease therapies are known to those of skill in the art.

Also encompassed by the present invention is a pharmaceutical composition, or compositions, comprising a therapeutically effective amount of the actCCP or inhCCP agents as described herein. The composition additionally can include a pharmaceutically acceptable medium, suitable as a carrier for the agent. The compositions can also include targeting agents to deliver the compositions to specific tumor sites.

The above and other features of the invention including various novel details of construction and combinations of parts, and other advantages, will now be more particularly described with reference to the accompanying drawings and pointed out in the claims. It will be understood that the particular methods and compositions embodying the invention are shown in the drawings and examples by way of illustration and not as a limitation of the invention. The principles and features of this invention may be employed in various and numerous embodiments without departing from the scope of the invention.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale, emphasis has instead been placed upon illustrating the principles of the invention. The patent or application file contains at least one drawing executed in color. Copies of this patent or application publication with color drawings will be provided by the Office upon request and payment of the necessary fee. Of the drawings:

FIG. 1. Complement Pathways (from Hajishengallis et al., 2017)

FIG. 2. Regulators of Complement Activity

FIG. 3. The C1q/TNF family

FIG. 4. Effect of scaffolds on apparent affinity for a target

FIG. 5. The design of CCP constructs showing effector, linker scaffold and targeting domains

FIG. 6. Examples of complement control protein domains (shown in magenta and green) attached by a linker to the C4BP scaffold

FIG. 7. Examples of complement control protein domains (shown in magenta and green) attached by a linker to the PD-1 scaffold

FIG. 8. Example of using the C1q globular domain for CCP constructs

FIG. 9. Examples of CCP constructs using antigen or receptor specific targeting scaffolds

FIG. 10. List of sequences described in the application

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The publications discussed throughout the text are provided solely for their disclosure prior to the filing date of the present application. Nothing herein is to be construed as an admission that the inventors are not entitled to antedate such disclosure by virtue of prior disclosure.

As discussed briefly above, the modern complement system uses multiples activators and regulators to identify threats both from invaders and from self. Many of these elements arose from an ancient set of building blocks that were duplicated and adapted to make new enzymes, activators, regulators and receptors (Hajishengallis et al., 2017). The common ancestor of eumetazoa used a system based only on complement component 3 (C3) convertase, factor B (FB) protease and the mannan binding lectin serine peptidase (MASP) activator (Nonaka, 2014). The system's earliest regulators, dating back to teleost fish, were membrane cofactor protein (MCP) and Factor I (FI) (Li et al., 2017). In the original schema, MASP, activated by non-self carbohydrates, cleaved FB leading to the proteolytic activation of the C3 Convertase, which was subsequently inactivated by FI bound to MCP. From this start evolved the classical pathway of complement activation initiated by antigen-bound antibody and the “always on” intravascular alternative pathway for labelling invaders that lack the ability to deactivate the cascade. The alternative pathway is self-amplifying and magnifies the response initiated by the other C3 convertases.

Labeling of surfaces by the complement pathway involves the covalent attachment of C3 and C4 non-specifically through a thioester bond to carbohydrates and proteins (Law and Dodds, 1997). It represents the first two signal system for regulating immune responses, preceding that for the adaptive immune system (Baxter and Hodgkin, 2002). Signal 1 is provided by the tags that are either immunostimulatory or immunosuppressive. Signal 2 is due to the C3a and C5a complement fragments released upon the initial cleavage of C3 and complement component 5 (C5) after activation of the system. These soluble peptides diffuse and generate a gradient that recruits and activates the appropriate effector cells to the site of the initial attack. The combination of signal 1 and signal 2 decides the nature of the immune response.

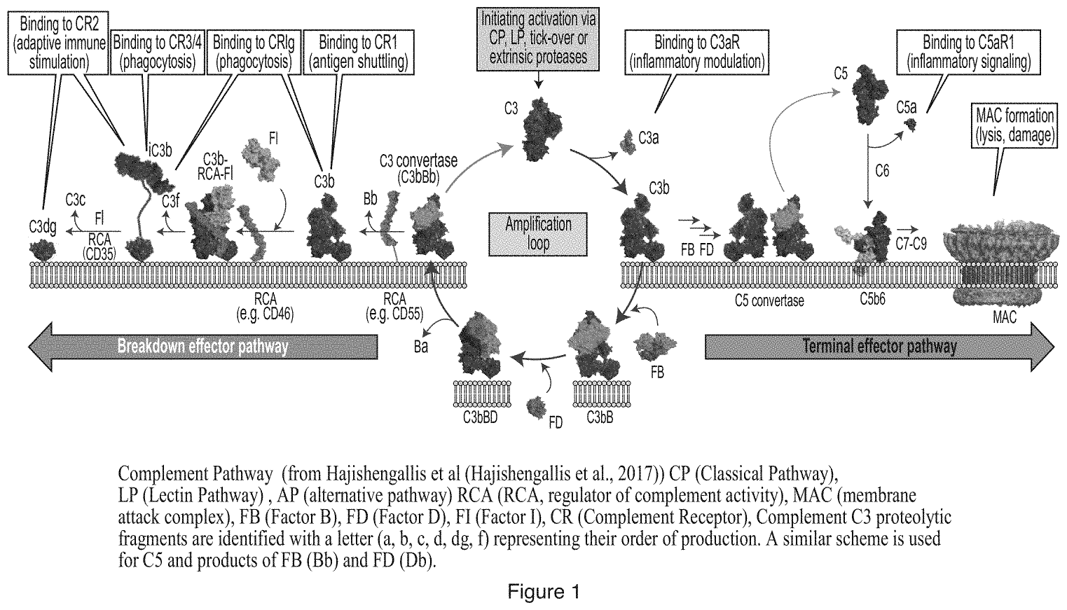

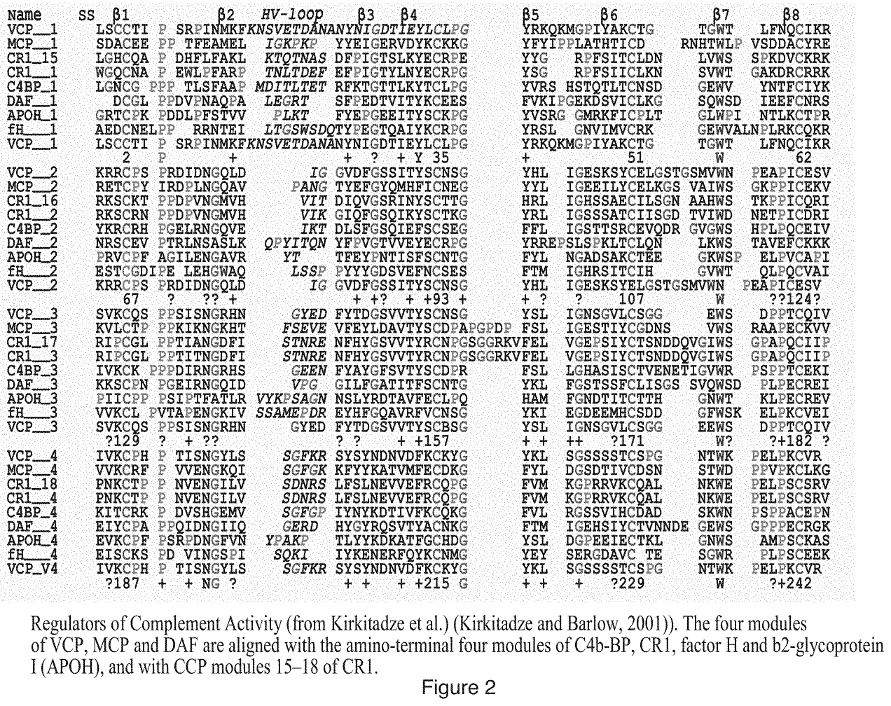

Regulators of complement activation (RCA) arose through the duplication and modification of the MCP complement control protein modules (CCP, known also as sushi domains short consensus repeats (SCR)) (Makou et al., 2015) (FIGS. 1 and 2, Tables 1-3). They consist of up to eight strands: β-strand 1, includes the N-terminus and Cystine I; β-strand 2 follows the consensus glycine at 8-10 positions beyond position Cystine I; β-strands 3, 4 and 5 occur within a ‘hXhGXXhXhXCIIXXG↓hXhXG’ motif (SEQ ID NO:1) (h is a hydrophobic residue and ↓ is a possible insertion; β-strand 6 precedes (and may include) Cystine III; β-strand 7 includes the consensus tryptophan; and β-strand 8 includes CysIV and the residues on either side (Makou et al., 2015). Many CCP-containing proteins have multiple binding sites for complement that often span neighboring CCPs (Makou et al., 2015). CCPs are present in a number of human RCA proteins including Factor H (FH), MCP (also known as CD46), decay-accelerating factor (DAF, CD55), complement receptors types 1 (CR1) and 2 (CR2), and the C4b-binding protein (C4b-BP). Many viruses also have incorporated RCA to help regulate host response that include the vaccinia and variola virus control proteins (VCP) (Kirkitadze and Barlow, 2001) (FIG. 2). Human RCAs cluster into two groups on chromosome one, each with different functionality (Krushkal et al., 2000). DAF accelerates decay and inactivation of convertase complexes (called decay-accelerating activity (DAA)) while others accelerate the Factor I cleavage of membrane bound C3b and C4b to inactivate them (referred to as cofactor activity (CA)). RCAs exist with different specificity for each pathway (Table 3).

Multiple adjacent CCPs in each protein combine to produce such activities. The number of CCPs in each protein can vary from 3 to the 30 present in CR1 (Krushkal et al., 2000). The longer repeats themselves arise through exon duplication, shuffling, recombination and gene conversion of ancestral genes (Krushkal et al., 2000). Modern day recombination events are associated with disease (Chen et al., 2016; Togarsimalemath et al., 2017). Many RCAs have multiple binding sites for target proteins, often with different specificities and activities. For example, in CR1, CCP 1-3 (site 1), CCP8-10 (site 2) and CCP15-17 (site 3) are repeats of each other (Krych-Goldberg and Atkinson, 2001). Site 1 has high DAA for C3 convertases but low CA, while site 2 and 3 are highly homologous with high CA but low DAA. The stabilities of CCP modules are often context dependent and influenced by contacts with neighboring modules (Kirkitadze and Barlow, 2001; Schmidt et al., 2016).

The specific complement control protein domains (CCP) are derived from RCA or Complement receptors and vary in their ability to promote decay accelerating activity (DAA) of convertases from the lectin pathway (LP), the alternative pathway (AP) or the classical pathway (CP), or to promote decay of C3 and C4 proteolytic fragments by acting as a cofactor (CA) for Factor I or to prevent activation of a convertase. As shown in Table 3, the score of 3 indicates high DAA or CAA activity with 0 representing no activity. The normal site of action of each activity is given. VSIG4 (amino acid residues 18-137) has an immunoglobulin-like motif with residues numbered by reference to Q9Y279 (uniprot.org). (See Table 1).

TABLE 1
Complement Regulators.

| | CP | AP | LP | C5 | AP | | | | Normal | |
|---|---|---|---|---|---|---|---|---|---|---|
| Ligand | DAA | DAA | DAA | DAA | Block | CoF | C3d | Site | Function | Reference |
| VSIG4 | 0 | 0 | 0 | 0 | 3 | 0 | No | Membrane | Inhibitory | [9] |
| (residues 18-137) | | | | | | | Binding | | | |
| CR1 site 1 | 3 | 3 | 3 | Site 1 | 0 | 1 | Cleavage | Membrane | Stimulatory | [8]} |
| (CCP 1-3) | | | | plus | | | Product | | | |
| | | | | Site 3 | | | | | | |
| CR1 sites 2, 3 | 1 | 1 | 1 | Site 1 | 0 | 3 | Cleavage | Membrane | Stimulatory | [8]} |
| CCP 5-8, 15-17 | | | | plus | | | Product | | | |
| | | | | Site 3 | | | | | | |
| CR2 (CCP1-2) | 0 | 0 | 0 | 0 | 0 | | Binds C3d | Membrane | Stimulatory | [10] |
| C3c/iC3b | 0 | 0 | 0 | 0 | 0 | 0 | Not a | Membrane | Inhibitory | [9] |
| | | | | | | | Product | | | |
| C4BP | 3 | 1 | 3 | 1 | 0 | 3 | Not a | Plasma | Inhibitory | [11] |
| (CCP 1-4) | | | | | | | Product | | | |
| CFH | 0 | 3 | 0 | 3 | 0 | 3 | Not a | Plasma | Inhibitory | [12] |
| (CCP 1-4, 1-5) | | (CCP 1-5) | | | | (CCP 1-4) | Product | | | |
| CFH5 | 0 | 0 | 0 | 3 | 0 | 0 | Note | Plasma | Inhibitory | [12] |
| (CCP3-9) | | | | | | | Product | | | |
| MCP | 0 | 0 | 0 | 0 | 0 | 3 | Not a | Membrane | Protective | [13] |
| (CCP 1-4) | | | | | | | Product | | | |
| DAF | 3 | 3 | 3 | 0 | 0 | 0 | Not a | Plasma | Inhibitory | [13] |
| (CCP 2-4) | | | | | | | Product | | | |

Other CCP proteins have both DAA and CA but differ in their convertase specificity and whether they act at surfaces or in solution (Table 2). They also differ in their affinity for a particular target (Forneris et al., 2016). Mutations of a single residue can also change activity (Forneris et al., 2016). For example, the mutation of Glutamine 1022 to Histidine (Q1022H) in CR1 CCP15-17 region increases CR1 binding affinity to C4b but not to C3b (Birmingham et al., 2003).

TABLE 2
Regulators of Complement Activation (RCA) (from
Schmidt [14]) .DAA (Decay accelerator Activity),
CA (Cofactor Activity), CP (Classical Pathway),
LP (Lectin Pathway), AP (alternative pathway)

| | Regulatory activity | | Regulated | Main regulatory |
|---|---|---|---|---|
| Regulator | Decay | Cofactor | pathway | compartment |
| CR1 | DAA | CA | CP/LP & AP | Surface |
| DAF | DAA | — | CP/LP & AP | Surface |
| MCP | — | CA | CP/LP & AP | Surface |
| C4BP | DAA | CA | CP/LP | Fluid/surface |
| Factor H | DAA | CA | AP | Fluid/surface |
| FHL-1 | DAA | CA | AP | Fluid/surface |

| | | | |
|---|---|---|---|
| Factor I | Protease for | CP/LP & AP | Fluid (on surface |
| | degradation of C3b | | only in conjunction |
| | or C4b in presence | | with cofactor) |
| | of a cofactor | | |

RCA have acquired other domains that target them, such as the (glycosylphosphatidylinositol) GPI anchor found in DAF (Shichishima, 1995) or allow their assembly into higher order structures, as shown for the oligomeric domain of C4BPα and C4BPβ (Hofmeyer et al., 2013). These particular adaptations appear in mammalian clades (Nakao and Somamoto, 2016), indicating that evolution of this ancient system remains a work in progress.

Complement protein receptors (CR) control the host response (Table 3) (Zipfel and Skerka, 2009). They have different specificity for complement proteolytic fragments. Certain receptors such as v-set and immunoglobulin domain containing 4 (VSIG4 also known as CRIg) bind surfaces containing C3c and inhibit complement convertases and proteases while others like CR3 bind inactivated C3b to promote phagocytosis of dead cells to terminate responses. CR2 binds C3d, the end-product of C3b proteolysis, and stimulates immune responses. CR1 is the only receptor that promotes formation of C3d. The interaction of CR1 with C3b differs from all other RCA as it does not block the FI cleavage site required for C3d production (Forneris et al., 2016; Krych-Goldberg and Atkinson, 2001). Overall, the final product of C3 proteolysis decides whether signal 1 is immunosuppressive or immunostimulatory (Zipfel and Skerka, 2009). (See Table 3).

TABLE 3
Complement Receptors (from [15])
Surface bound regulators and effectors

| | | | | | |
|---|---|---|---|---|---|
| CR1 | CD35 and | C3 | C3b, iC3b, C4b | Many nucleated cells | Clearance of immune complexes, |
| | immune | | and C1q | and erythrocytes, B cells, | enhancement of phagocytosis and |
| | [symbol] | | | leukocytes, monocytes and | regulation of C3 breakdown |
| | receptor | | | follicular dendritic cells | |
| CR2 | CD21 and | C3 | C3dg, C3d and | B cells, T cells and follicular | Regulation of B cell functions, B cell |
| | Epstein-Barr | | iC3b | dendritic cells | co-receptor and retention of C3d |
| | [symbol] receptor | | | | tagged immune complexes |
| CR3 | MAC1, | C3 | iC3b and factor | Monocytes, macrophages, | iC3b enhances the contact of |
| | CD11b-CD18 | | H | neutrophils, natural killer cells, | [symbol] , resulting in |
| | and αMβ2 | | | eosinophils, myeloid cells, | phagocytosis and adhesion by CR3 |
| | integrin | | | follicular dendritic cells, CD4⁻ | |
| | | | | T cells and CD8⁺ T cells | |
| CR4 | CD11c-CD1 [symbol] | C3 | iC3b | Monocytes and macrophages | iC3b-mediated phagocytosis |
| | and αXβ2 integrins | | | | |
| CRIg | VSIG4 | C3 | C3b, iC3b and | Macrophages | iC3b-mediated phagocytosis and |
| | | | C3c | | inhibition of alternative pathway |
| | | | | | activation |
| CD46 | MCP | C3 | C3b and C4b | All cells except erythrocytes | C3 degradation, cofactor for factor I |
| | | | | | and factor H, and effector for T cell |
| | | | | | maturation |
| CD55 | DAF | C3 | C4b2b and | GPI anchor expression by | Acceleration of C3 convertase decay |
| | | | C3bBb | most cell types, including | |
| | | | | erythrocytes, epithelial cells | |
| | | | | and endothelial cells | |
| CD5 [symbol] | [symbol] | TCC | C8 and TCC | GPI anchor expression by | Inhibition of TCC assembly and |
| | | | | erythrocytes and most | formation |
| | | | | nucleated cells, including | |
| | | | | renal cells | |

[symbol]

indicates data missing or illegible when filed

Various protein-based therapeutic strategies exist for regulating the complement system. These aim at reducing complement driven inflammation, primarily those associated with autoimmunity and transplantation rejection (Ricklin et al., 2018). A cut-down version of CFH that replaces CCP5-18 of CFH with a linker sequence and called mini-FH shows improved activity (Harder et al., 2016; Schmidt et al., 2013). A fusion of CR2 (CCP1-4) and CFH (CCP1-5) called TT30 is designed to target CA and DAA of CFH to cells where the complement split product C3b and C3d are deposited (Fridkis-Hareli et al., 2011). Another hybrid from DAF (CCP2-3) and MCP (CCP3-4) has robust CA for C3b and C4b and DAA for classical and alternative pathway C3 convertases (Panwar et al., 2019). These differ from the constructs described here in lacking a scaffold domain and the absence of a targeting ligand other than that due to the CCP domains. Consequently, there are important limits on optimizing of their pharmacokinetics and pharmacodynamics properties.

Another therapeutic approach has been to include complement receptors in the therapeutic. In one example, VSIG4 (CRIg residues 19-1370 is used to target CFH (CCP1-5, residues 19-323) to inflamed tissues (Hu et al., 2018; Qiao et al., 2014). With another approach, the C4BP scaffold is used to target immune complexes to the liver by creating a reagent that uses CR1 to capture the complexes and a single-chain Fv anti-Rh(D) to target the bound immune complexes to erythrocytes (Oudin et al., 2000). The therapeutic represents the fusion of the entire extracellular domain of complement receptor CR1 to C4BPα scaffold (C-terminal 167 bp fragment), while the Fv anti-Rh(D) is fused to C4BPβ. The hybrid scaffold resulted in 6 C4BPα chains bearing the CR1 domains and only 1 C4BPβ chain with the Fv protein (Hofmeyer et al., 2013). The C4BPα scaffold has also been employed to generate vaccines that present antigenic multimers to the immune system (Brune et al., 2017; Ogun et al., 2008) and for increasing the avidity of peptide ligands for their target (Maass et al., 2015; Valldorf et al., 2016).

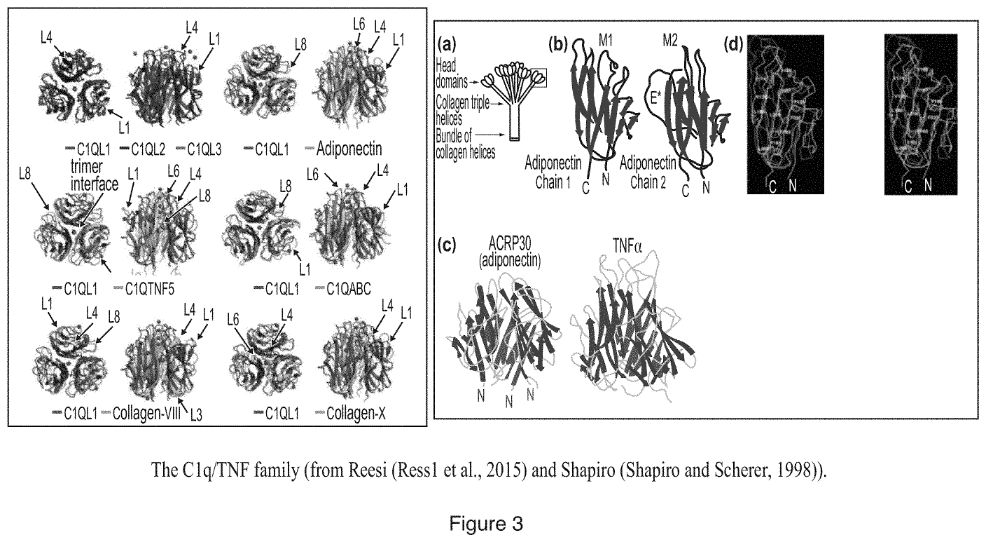

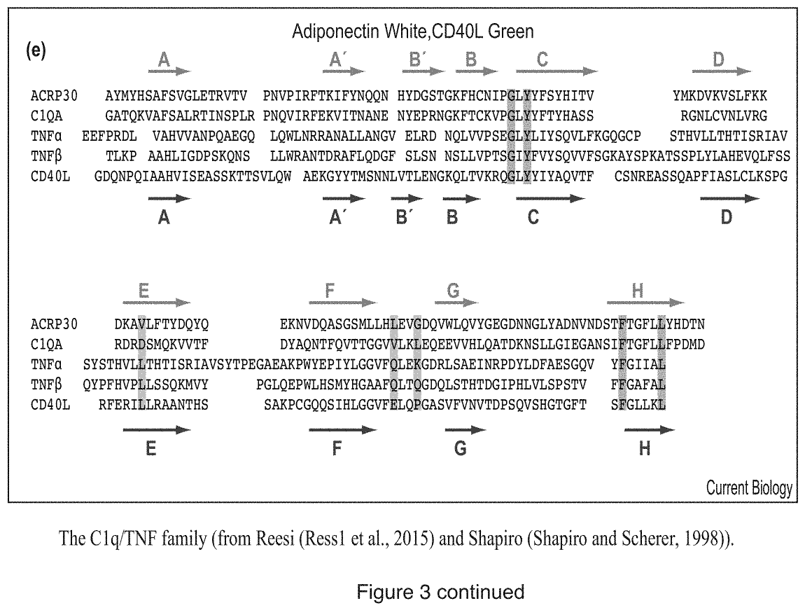

Other potential scaffolds exist that permit targeting of CCPs to different receptors. The use of these scaffolds as described in this invention is novel. For example, the C1q and TNF family includes not only complement C1q but other members like TNFα (TNF), 4-1BB (TNFRSF9), Apo2L/TRAIL (TNFSF10), LTα (LTA), RANKL(TNFSF11), LIGHT (TNFSF14) and CD40L (Kishore et al., 2004; Shapiro and Scherer, 1998; Tom Tang et al., 2005). The C1q-like globular domains form from three separate chains. Expression of these domains as a single chain retains (Moreau et al., 2016) their binding specificity and potentially enables fusion of CCPs to both amino- and carboxy-termini. FIG. 3 presents a sampling of this family along with structure based alignments.

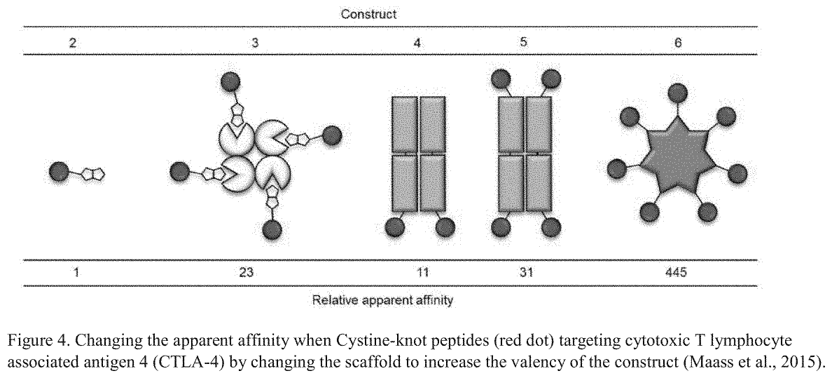

It is also possible to use scaffolds to alter the avidity of binding to a targeted receptor. FIG. 4 provides an example of how a change of valency changes apparent affinity. For example, a peptide fused to the C4BP oligomer domain has a 445 times higher apparent affinity for its target than a monomer. The approach permits use of lower affinity targeting elements to target sites where the receptors are most numerous (Grochmal et al., 2013) as is characteristic of many immunological disease states.

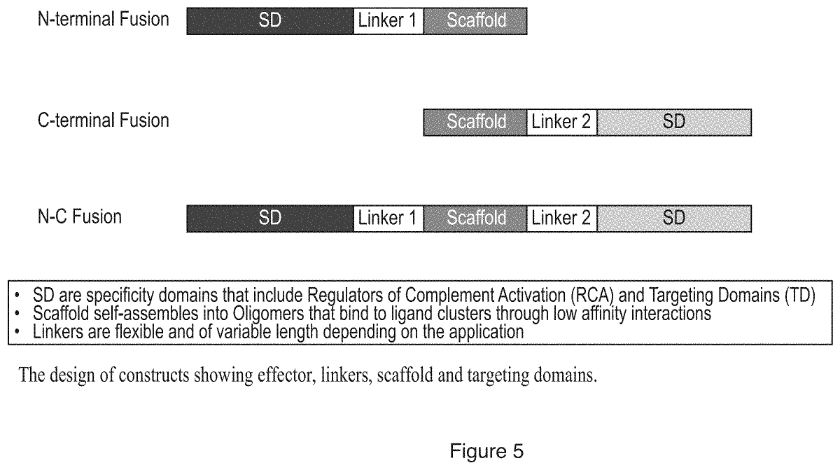

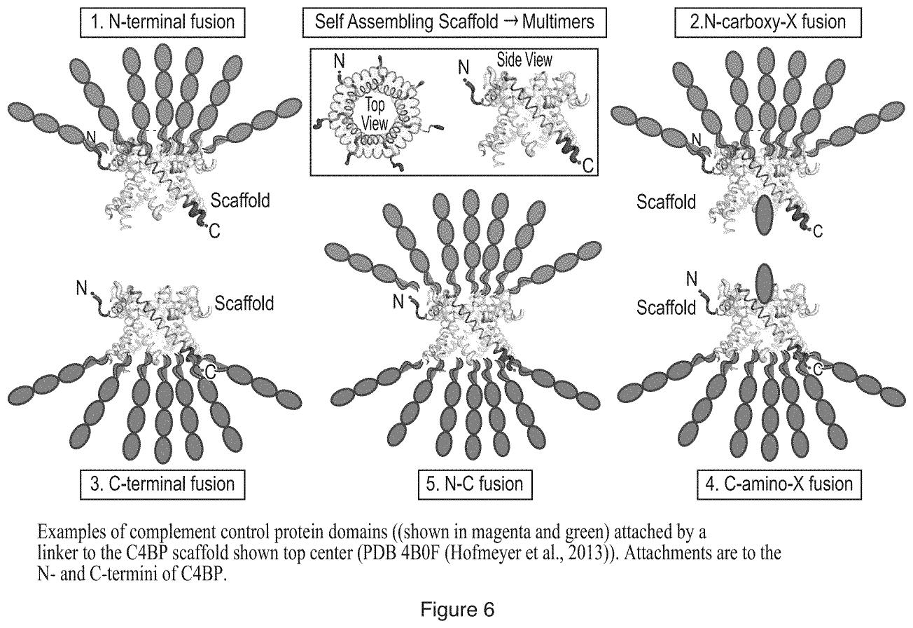

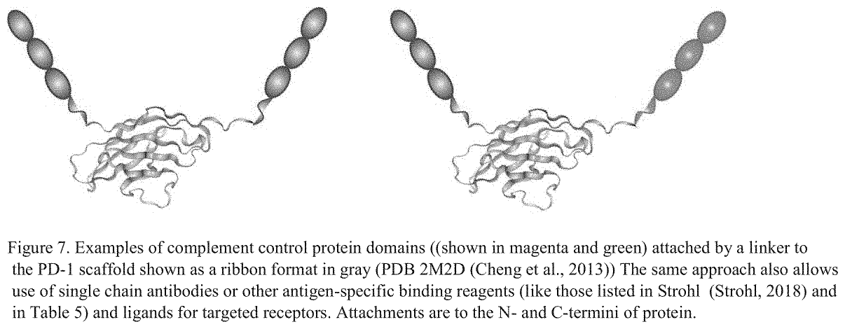

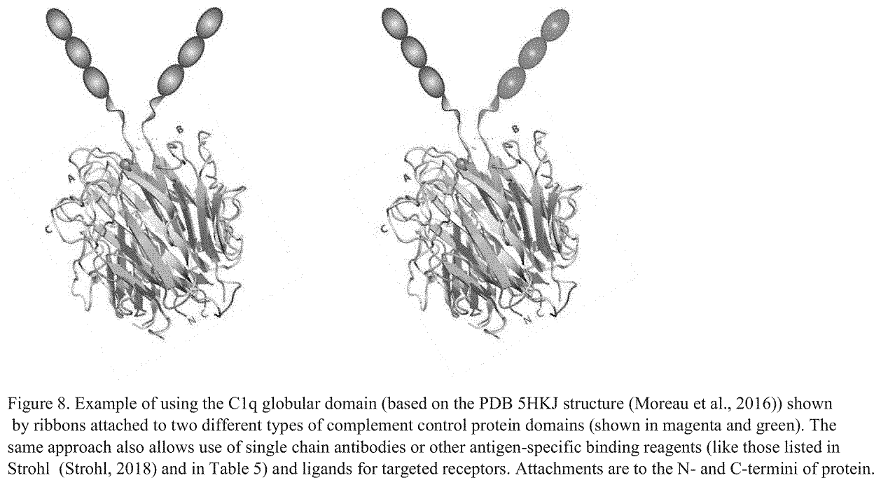

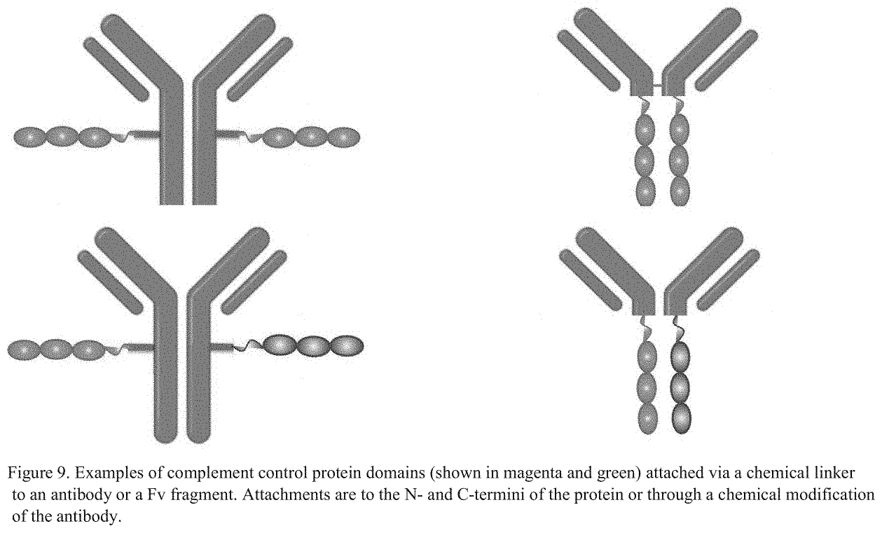

There are a number of suitable scaffolds that can be used in different ways (FIGS. 5 to 9), following the designs in FIG. 5. The C4BP framework is compatible with a large number of designs that vary the type and number of CCPs attached and allow incorporation of targeting ligands (FIG. 6). A similar strategy the extracellular domains of natural ligands also enables use of both termini to deliver effectors to sites of action. One example is the use of PD1 fusions to target cells bearing PD-L1 and PD-L2 receptors, where binding affinity can be modulated using PD1 mutations (Li et al., 2018; Maute et al., 2015)(FIG. 7). Another approach uses C1q/TNF family single chain constructs as scaffolds (FIG. 8). Single chain antibody, nanobody, duabody, affibody, repebody scaffolds or antigen-specific scaffolds ((Strohl, 2018) and Table 4) allow targeting of CCP domains fused to them (FIG. 9).

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, exemplary methods, and materials are described herein.

General texts, which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning-A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”). Examples of protocols sufficient to direct persons of skill through in vitro amplification methods, including the polymerase chain reaction (PCR), the ligase chain reaction (LCR), Q.beta.-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13: 563-564. Improved methods for cloning in vitro amplified nucleic acids are described in Wallace et al., U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated.

The terms “cell”, “exosome” and “extra-cellular vesicle” are used in reference to closed surfaces bearing CCPs with or without antigens and are used without regard to their contents or to their species.

The terms “vector”, “vector construct” and “expression vector” mean the vehicle by which a DNA or RNA sequence (e.g. a foreign gene) can be introduced into a host cell, so as to transform the host and promote expression (e.g. transcription and translation) of the introduced sequence. Vectors typically comprise the DNA of a transmissible agent, into which foreign DNA encoding a protein is inserted by restriction enzyme technology. A common type of vector is a “plasmid”, which generally is a self-contained molecule of double-stranded DNA that can readily accept additional (foreign) DNA and which can readily introduced into a suitable host cell. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g., antibiotic resistance, and one or more expression cassettes.

In one embodiment, the viral vector can be a replication competent retroviral vector capable of infecting only replicating tumor cells with particular mutations. In one embodiment, a replication competent retroviral vector comprises an internal ribosomal entry site (IRES) 5′ to the heterologous polynucleotide encoding, e.g., a cytosine deaminase, miRNA, siRNA, cytokine, receptor, antibody or the like. When the heterologous polynucleotide encodes a non-translated RNA such as siRNA, miRNA or RNAi then no IRES is necessary, but may be included for another translated gene, and any kind of retrovirus (see below) can be used. In one embodiment, the polynucleotide is 3′ to an ENV polynucleotide of a retroviral vector. In one embodiment the viral vector is a retroviral vector capable of infecting targeted tumor cells multiple times (5 or more per diploid cell).

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. A polynucleotide or polypeptide is expressed recombinantly, for example, when it is expressed or produced in a foreign host cell under the control of a foreign or native promoter, or in a native host cell under the control of a foreign promoter. These recombinantly produced polypeptides can then be purified and administered as therapeutics.

The terms “gene editing” or “gene editing techniques” as described herein can include RNA-mediated interference (referred to herein as RNAi, or interfering RNA molecules), or Short Hairpin RNA (shRNA) or CRISPR-Cas9 and TALEN. See e.g., Agrawal. N. et al., Microbiol Mol Biol Rev. 2003 December; 67(4): 657-685; Moore, C. B., et al. Methods Mol Biol. 2010; 629: 141-158; Doudna, J A. and Charpentier, E. Science vo. 346, 28 Nov. 2014; Sander, J. D. and Joung, K. Nature Biotech 32, 347-355 (2014); U.S. Pat. No. 8,697,359; Nemudryo, A. A. ACTA Naturae vol. 6, No. 3(22)2014. Anti-sense RNA can also be used. (Gleave, M. and Monia, B., Nature Reviews Cancer 5, 468-479 (June 2005)). The term “gene therapy” generally means a method of therapy wherein a desired gene/genetic sequence is inserted into a cell or tissue (along with other sequences necessary for the expression of the specific gene). See, for example, genetherapynet.com for description of gene therapy techniques.

The term “subject” as used herein can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “cancer” or “tumor” includes, but is not limited to, solid tumors and blood borne tumors. These terms include diseases of the skin, tissues, organs, bone, cartilage, blood and vessels. These terms further encompasses primary and metastatic cancers. Biomarkers identifying the expression of C3, C3b, C3c, C3d, C4, C4d, C5, C3aR1, C5aR1, C5aR2, C1R, C1RL, CR2, C1QBP, CD46, CD55, CD59, or LAIR1 in tumors provide one means of selecting patients for treatment, whether the biomarker is detected by RNA expression, antibody or other reagents that allow quantitation of these molecules.

The term “antigen” is defined as any molecule that a T-Cell or B-Cell receptor has specificity for, or any molecule bound by Natural Killer Cells or other Innate Cells that specifically targets their effector function such as cytotoxic killing of cells, release of growth factors, lymphokines or cytokines. (Microbiology and Immunology On-line, Edited by Richard Hunt, PhD; www.microbiologybook.org/mayer/antigens2000)

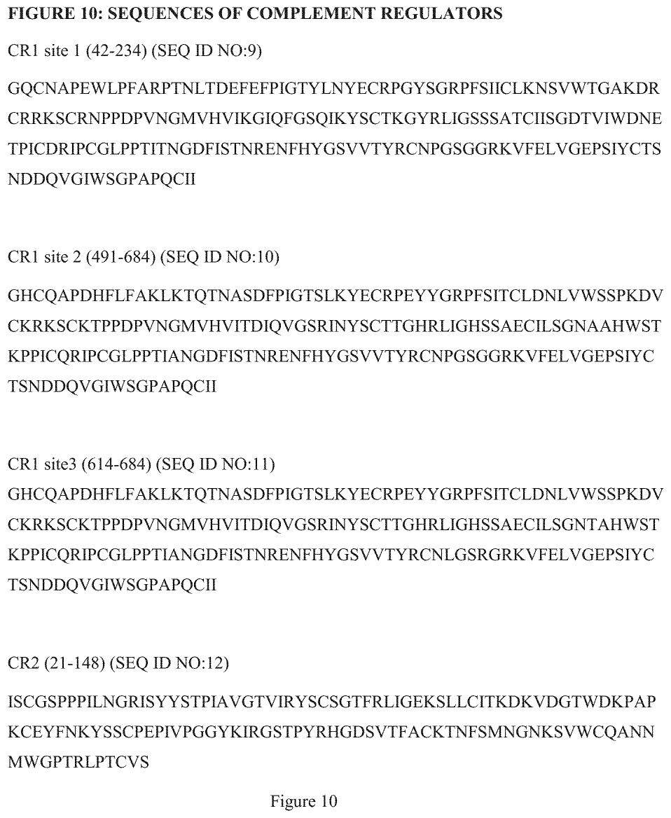

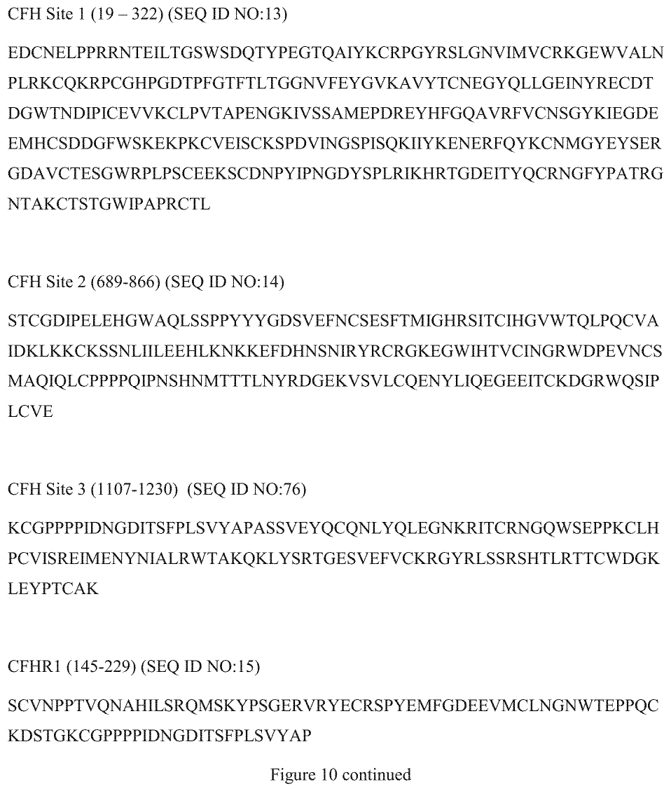

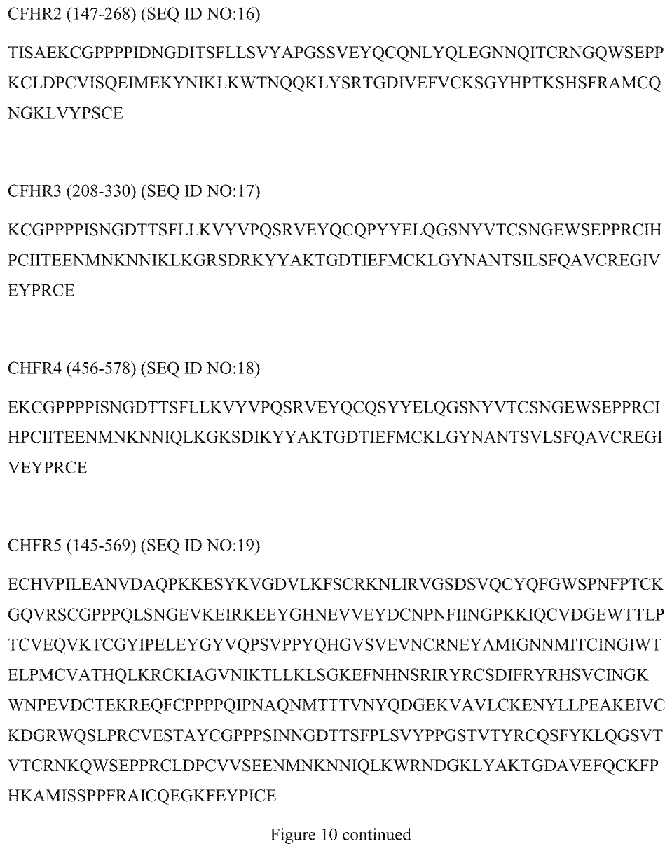

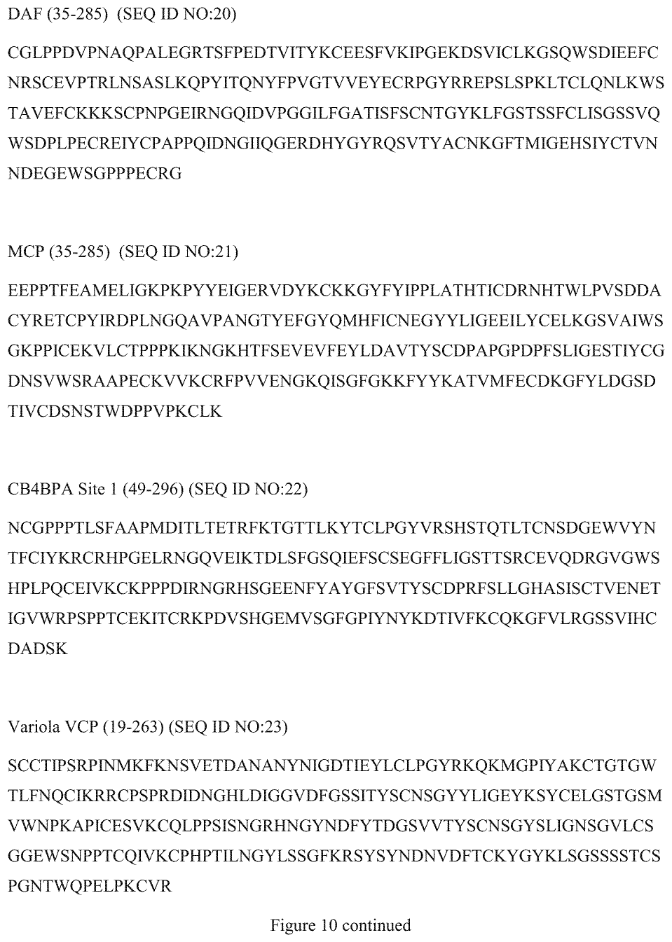

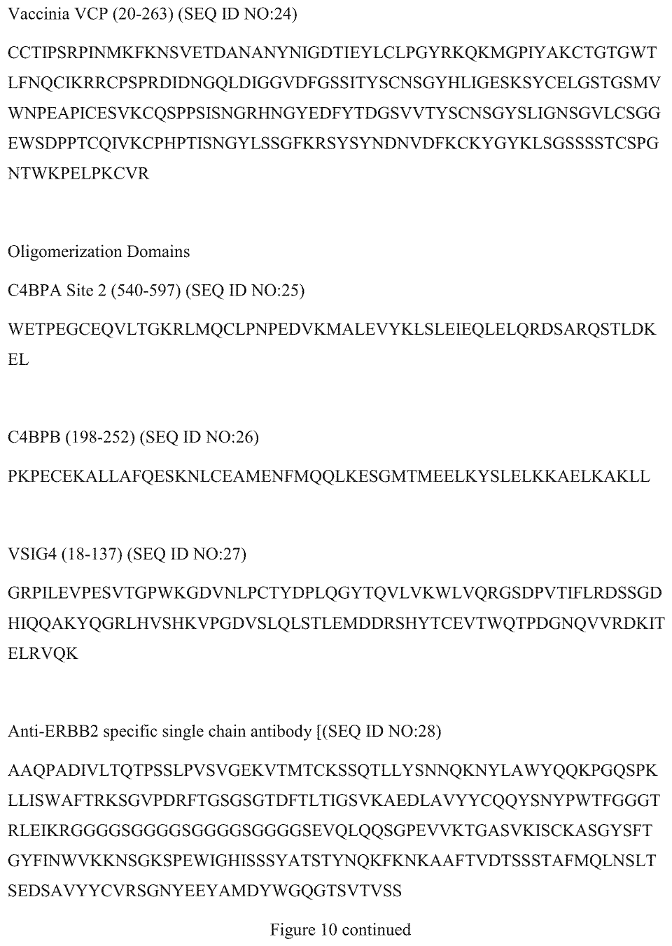

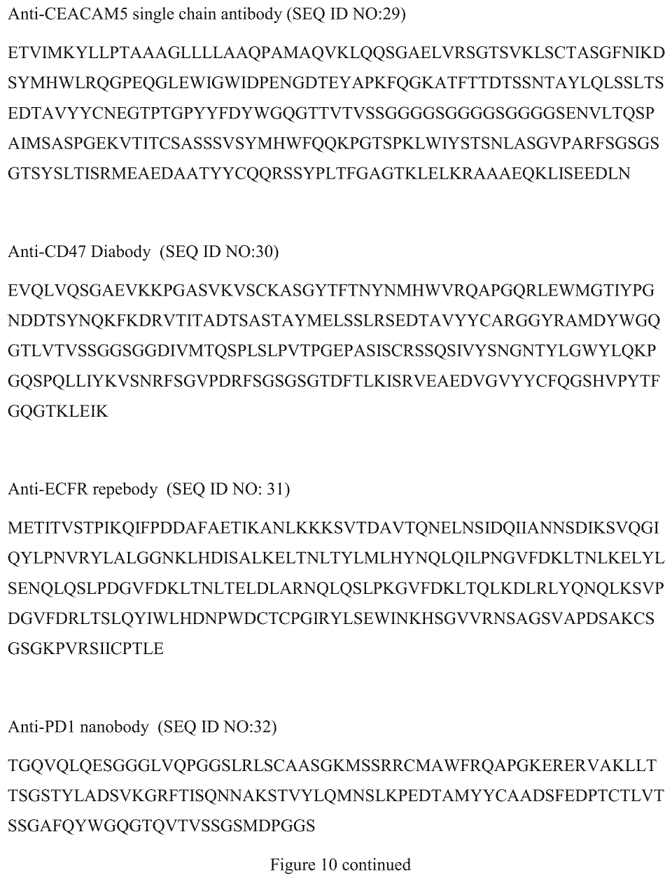

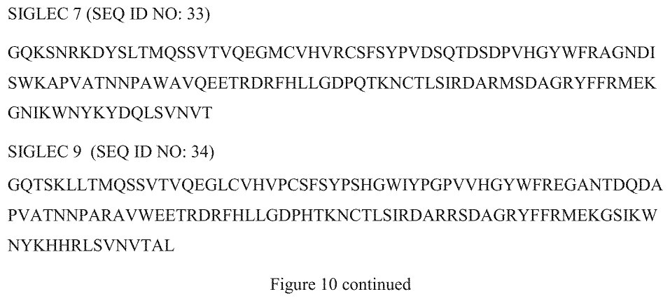

The term “CCP” refers to complement control protein domains (references 2, 27). For the purposes of this invention, it specifically refers to entities listed in FIG. 10 that will activate immune responses (“actCCP”) or inhibit them (“inhCCP”) according to the particular properties of the CCP, either wildtype or after mutation of specific residues.

The methods and compositions of the present invention may be used to treat any type cancerous tumor or cancer cells. Such tumors/cancers may be located anywhere in the body, including without limitation in a tissue selected from brain, colon, urogenital, lung, renal, prostate, pancreas, liver, esophagus, stomach, hematopoietic, breast, thymus, testis, ovarian, skin, bone marrow and/or uterine tissue. Cancers that may treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor, meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The methods and compositions of the present invention may be used to treat any type of disease/disorder associated with the immune system of the subject, and in particular, any inflammatory diseases. Examples of disorders associated with inflammation include: acne vulgaris; asthma; autoimmune diseases; auto-inflammatory diseases; celiac disease; cellulitis; chronic prostatitis; colitis; diverticulitis; glomerulonephritis; hypersensitivities; inflammatory bowel diseases; interstitial cystitis; mast cell activation syndrome; mastocytosis; otitis; pelvic inflammatory disease; psoriasis; ischemic injury such as reperfusion injury, rheumatoid arthritis; rhinitis; sarcoidosis; transplant rejection and vasculitis.

A “therapeutically effective” amount as used herein refers to an amount sufficient to have the desired biological effect (for example, an amount sufficient to express the CCPs to produce the desired effect on the underlying disease state (for example, an amount sufficient to inhibit tumor growth in a subject, produce an immune response to an antigen or to inhibit autoimmune disease) in at least a sub-population of cells in a subject at a reasonable benefit/risk ratio applicable to any medical treatment. Determination of therapeutically effective amounts of the agents used in this invention, can be readily made by one skilled in the art, by the use of known techniques and by observing results obtained under analogous circumstances. The amounts/dosages may be varied depending upon the requirements of the subject in the judgment of the treating clinician; the severity of the condition being treated and the particular composition being employed. In determining the therapeutically effective amount, a number of factors are considered by the treating clinician, including, but not limited to: the specific disease state; pharmacodynamic characteristics of the particular agent and its mode and route of administration; the desired time course of treatment; the species being treated; its size, age, and general health; the specific disease involved; the degree of or involvement or the severity of the disease; the response of the individual patient; the particular agent administered; the mode of administration; the bioavailability characteristics of the preparation administered; the dose regimen selected; the kind of concurrent treatment (i.e., the interaction of the agent with other co-administered agents); and other relevant circumstances.

For example, as described herein, the amino acid sequence of the antigens can be truncated/mutated/altered to produce biologically active reagents or variants. Antigens can be large molecules (e.g., proteins, lipids, carbohydrates) or small molecules (e.g., synthetic inorganic, organometallic, or organic molecules) Such antigens can be synthesized in those skilled in the art, or otherwise produced, and evaluated for their biological and immunological activity. Variants or fusions of the antigens can specifically increase MHC binding to increase immunomodulation.

In certain embodiments, the agents described for use in this invention can be combined with other pharmacologically active compounds (“additional active agents”) or antigens (“antigens”) known in the art according to the methods and compositions provided herein. Additional active agents can be large molecules (e.g., proteins, lipids, carbohydrates) or small molecules (e.g., synthetic inorganic, organometallic, or organic molecules). In one embodiment, additional active agents independently or synergistically help to treat cancer.

For example, certain additional active agents are anti-cancer chemotherapeutic agents. The term chemotherapeutic agent includes, without limitation, platinum-based agents, such as carboplatin and cisplatin; nitrogen mustard alkylating agents; nitrosourea alkylating agents, such as carmustine (BCNU) and other alkylating agents; antimetabolites, such as methotrexate; purine analog antimetabolites; pyrimidine analog antimetabolites, such as fluorouracil (5-FU) and gemcitabine; hormonal antineoplastics, such as goserelin, leuprolide, and tamoxifen; natural antineoplastics, such as taxanes (e.g., docetaxel and paclitaxel), aldesleukin, interleukin-2, etoposide (VP-16), interferon alfa, and tretinoin (ATRA); antibiotic natural antineoplastics, such as bleomycin, dactinomycin, daunorubicin, doxorubicin, and mitomycin; and vinca alkaloid natural antineoplastics, such as vinblastine and vincristine or agents targeted at specific mutations within tumor cells.

Further, the following drugs may also be used in combination with an antineoplastic agent, even if not considered antineoplastic agents themselves: dactinomycin; daunorubicin HCl; docetaxel; doxorubicin HCl; epoetin alfa; etoposide (VP-16); ganciclovir sodium; gentamicin sulfate; interferon alfa; leuprolide acetate; meperidine HCl; methadone HCl; ranitidine HCl; vinblastin sulfate; and zidovudine (AZT). For example, fluorouracil has recently been formulated in conjunction with epinephrine and bovine collagen to form a particularly effective combination.

Still further, the following listing of amino acids, peptides, polypeptides, proteins, polysaccharides, and other large molecules may also be used in conjunction with the invention: checkpoint inhibitors that target for example, PD-1 and CTLA-4, interleukins 1 through 37, including mutants and analogues; interferons or cytokines, such as interferons .alpha., .beta., and .gamma.; hormones, such as luteinizing hormone releasing hormone (LHRH) and analogues and, gonadotropin releasing hormone (GnRH); growth factors, such as transforming growth factor-.beta. (TGF-.beta.), fibroblast growth factor (FGF), nerve growth factor (NGF), growth hormone releasing factor (GHRF), epidermal growth factor (EGF), fibroblast growth factor homologous factor (FGFHF), hepatocyte growth factor (HGF), and insulin growth factor (IGF); tumor necrosis factor-.alpha. & .beta. (TNF-.alpha. & .beta.); invasion inhibiting factor-2 (IIF-2); bone morphogenetic proteins 1-7 (BMP 1-7); somatostatin; thymosin-.alpha.-1; .gamma.-globulin; superoxide dismutase (SOD), complement factors; anti-angiogenesis factors; antigenic materials; and pro-drugs.

Chemotherapeutic agents for use with the compositions and methods of treatment described herein include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

The compositions and methods of the invention can comprise or include the use of other biologically active substances, including therapeutic drugs or pro-drugs, for example, other chemotherapeutic agents or antigens useful for cancer vaccine applications. Various forms of the chemotherapeutic agents and/or additional active agents may be used. These include, without limitation, such forms as uncharged molecules, molecular complexes, salts, ethers, esters, amides, and the like, which are biologically active.

The agents and substances described herein can be delivered to the subject in a pharmaceutically suitable, or acceptable or biologically compatible carrier. The terms “pharmaceutically suitable/acceptable” or “biologically compatible” mean suitable for pharmaceutical use (for example, sufficient safety margin and if appropriate, sufficient efficacy for the stated purpose), particularly as used in the compositions and methods of this invention.

The compositions described herein may be delivered by any suitable route of administration for treating the cancer, including orally, nasally, transmucosally, ocularly, rectally, intravaginally, parenterally, including intramuscular, subcutaneous, intramedullary injections, as well as intrathecal, direct intraventricular, intravenous, intra-articular, intra-sternal, intra-synovial, intra-hepatic, through an inhalation spray, or other modes of delivery known in the art.

The nucleic acid sequence for C3, including iC3b, C3d and C3dg can be found e.g., in Proc. Natl. Acad. Sci. USA, vol. 82, pp. 708-712, February 1985). The term “C3d” as used herein is intended to encompass both C3d and C3dg and the term “iC3b” is used to encompass “C3c”. The nucleic acid sequence for the C3aR can be found at “C3AR1 complement C3a receptor 1 [Homo sapiens (human)]” Gene ID: 719, www.ncbi.nlm.nih.gov/gene, updated on 6 Aug. 2017. The nucleic acid sequence for the C5a receptor can be found at “C5AR1 complement C5a receptor 1 [Homo sapiens (human)]” Gene ID: 728, www.ncbi.nlm.nih.gov/gene, updated on 29 Aug. 2017. The nucleic acid sequence for other genes can be found as listed below. CiR complement Cir [Homo sapiens (human)], Gene ID: 715, www.ncbi.nlm.nih.gov/gene, updated on 10 May 2019, C1RL complement Cir subcomponent like [Homo sapiens (human), Gene ID: 51279, www.ncbi.nlm.nih.gov/gene, updated on 10 May 2019, C5AR2 complement component 5a receptor 2 [Homo sapiens (human)], Gene ID: 27202, www.ncbi.nlm.nih.gov/gene, updated on 10 May 2019, CIQBP complement Clq binding protein [Homo sapiens (human)}, Gene ID: 708, www.ncbi.nlm.nih.gov/gene, updated on 10 May 2019, CR2 complement C3d receptor 2 [Homo sapiens (human)], Gene ID: 1380, www.ncbi.nlm.nih.gov/gene, updated on 10 May 2019, CD46 molecule [Homo sapiens (human)], Gene ID: 4179, www.ncbi.nlm.nih.gov/gene, updated on 10 May 2019, CD55 molecule (Cromer blood group) [Homo sapiens (human)], Gene ID: 1604, www.ncbi.nlm.nih.gov/gene, updated on 6 Sep. 2017, CD59 molecule (CD59 blood group) [Homo sapiens (human)], Gene ID: 966, www.ncbi.nlm.nih.gov/gene, updated on 10 May 2019 and LAIR1 leukocyte associated immunoglobulin like receptor 1 [Homo sapiens (human)], Gene ID: 3903, www.ncbi.nlm.nih.gov/gene, updated on 10 May 2019. The nucleic acid sequence for the proteases cathepsin L [Homo sapiens (human)], CTSL, Gene ID: 1514 and cathepsin S [Homo sapiens (human)], CTSS, Gene ID: 1520, can be found at www.ncbi.nlm.nih.gov/gene, updated on 10 May 2019. VSIG4 (Gene ID 11326, www.ncbi.nlm.nih.gov/gene/11326, updated on 10 May 2019); CR1 [Homo sapiens (human)], (Gene ID 1378, www.ncbi.nlm.nih.gov/gene/1378); CR2 [Homo sapiens (human)], (Gene ID 1380, www.ncbi.nlm.nih.gov/gene/1380, updated on 10 May 2019); LTA [Homo sapiens (human)], (Gene ID, 4049, www.ncbi.nlm.nih.gov/gene/40490, updated on 10 May 2019), TNFSF14 [Homo sapiens (human)], (Gene ID, 8740, www.ncbi.nlm.nih.gov/gene/8740, updated on 10 May 2019), TNFSF11 [Homo sapiens (human)], (Gene ID, 8600, www.ncbi.nlm.nih.gov/gene/8600, updated on 10 May 2019), TNFSF10 [Homo sapiens (human)], (Gene ID, 8743, www.ncbi.nlm.nih.gov/gene/8743, updated on 10 May 2019), TNFRSF9 [Homo sapiens (human)], (Gene ID, 3604, www.ncbi.nlm.nih.gov/gene/3604, updated on 10 May 2019), ERBB2 [Homo sapiens (human)], (Gene ID, 2064, www.ncbi.nlm.nih.gov/gene/2064, updated on 10 May 2019), ERBB3 [Homo sapiens (human)], (Gene ID, 2065, www.ncbi.nlm.nih.gov/gene/2065, updated on 10 May 2019), ERBB4 [Homo sapiens (human)], (Gene ID, 2066, www.ncbi.nlm.nih.gov/gene/2066, updated on 10 May 2019), KDR [Homo sapiens (human)], (Gene ID, 3791, www.ncbi.nlm.nih.gov/gene/3791, updated on 10 May 2019), FLT1 [Homo sapiens (human)], (Gene ID, 2321, www.ncbi.nlm.nih.gov/gene/2321, updated on 10 May 2019), FLT4 [Homo sapiens (human)], (Gene ID, 2324, www.ncbi.nlm.nih.gov/gene/2324, updated on 10 May 2019), EGFR [Homo sapiens (human)], (Gene ID, 1956, www.ncbi.nlm.nih.gov/gene/1956, updated on 10 May 2019), CEACAM5 [Homo sapiens (human)], (Gene ID, 1048, www.ncbi.nlm.nih.gov/gene/1048, updated on 10-Mav-2019), CD274 [Homo sapiens (human)], (Gene ID, 29126, www.ncbi.nlm.nih.gov/gene/29126, updated on 10 May 2019), CD47 [Homo sapiens (human)], (Gene ID, 961, www.ncbi.nlm.nih.gov/gene/961, updated on 10 May 2019), C4BPA [Homo sapiens (human)], (Gene ID, 722, www.ncbi.nlm.nih.gov/gene/722, updated on 10 May 2019), C4BPB [Homo sapiens (human)], (Gene ID, 725, www.ncbi.nlm.nih.gov/gene/725, updated on 10 May 2019).

For example, a gene editing technique to produce the CCP transcript within tumors can be used so that the protein product is targeted to the cell surface membrane as described in this invention (see e.g., U.S. Pat. No. 8,697,359 for a description of CRISPR techniques). Delivery of CRISPR/CAS9 with a sgRNAs to C3 (excluding the C3d sequence) and the nucleic acid sequences for C3d or C3d derived peptides, to a tumor cell can be provided by use of a viral vector. Delivery of CRISPR/CAS9 with a sgRNAs to C3 (excluding the C3c sequence) and the nucleic acid sequences for C3c or C3c derived peptides to a tumor cell along with other sequences necessary for targeting of the CCP transcripts that are introduced into cleavage sites during the process of repair, can be provided by use of a viral vector. A number of viral vectors have been used in humans and these can be used to transduce the genetic material in different cell types. Such methods are known to those of skill in the art. Means to target the vectors for specific delivery of the constructs to the tumor cells of interest are also known to those of skill. For example, genetically engineered vectors exist where the capsid is modified to contain ligands for receptors that facilitate viral entry onto a particular cell type. An example is given in FIG. 1. This construct also includes a reporter gene that allows efficiency of transduction of the virus into the tumor to be quantitated.

The above approaches can be combined with other cancer therapies including immune-modulators such as checkpoint inhibitor ligands for PD-1 CTLA-4, ICOS, OX40; reagents against C3a and C5a receptors; lymphokines, cytokines and their receptors and strategies designed to increase major and minor histocompatibility antigens. Additionally, the methods of the present invention can be combined with other standard cancer therapies such as radiotherapy and chemotherapy.

### EXAMPLES

The design of actCCP is to enrich target membranes for C3d or C4d, or both, by removing iC3b or iC4b or both, preventing formation of iC3b or iC4b or both, or promoting its conversion to C3d or C4d or both. actCCPs are engineered by fusing one, or more, of the same or different CR1 CCP or C3d or C4d to a scaffold. Localization of actCCP to target membranes is through the receptor binding properties of the scaffold or by receptor specific ligands fused/linked to the scaffold. In the case that the scaffold is receptor binding, actCCPs are fused to the amino- and/or carboxy-termini. In the case where the scaffold self-assembles into an oligomer, the actCCP may be fused to either the amino- or carboxy termini or to both. Alternatively, one actCCP can be fused to the amino-terminus and another to actCCP the carboxy-terminus. Alternatively, the actCCP may be fused to one terminus and targeting ligand to either, for example, Tumor Necrosis Factor Receptors, HER2/NEU, Vascular Endothelial Growth Factor Receptors, Epithelial Growth Factor Receptors, PD-L1, PD-L2 or C1q/TNF family members at the other terminus. Alternatively, the CR1 CCP s and targeting ligands to either Tumor Necrosis Factor Receptors, HER2/NEU, Vascular Endothelial Growth Factor Receptors, Epithelial Growth Factor Receptors, PD-L1, PD-L2 or C1q/TNF family members can be attached to a different subunit of the scaffold oligomer than the actCCP.

The design of inhCCP is to prevent C3 and/or C4 convertase activation and to prevent conversion by Factor I of iC3b or iC4b or both into C3d. or C4d or both. The inhCCP are engineered by fusing one or more of the same, or different, CCP derived from Factor H, MCP, DAF, C4BP or VSIG4 to a scaffold. Localization of inhCCP to target membranes is through the receptor binding properties of the scaffold or by receptor specific ligands fused to the scaffold. In the case that the scaffold is receptor binding, inhCCPs are fused to the amino- and carboxy-termini. In the case where the scaffold self-assembles into an oliogomer, the inhCCP may be fused to either the amino- or carboxy termini or to both. Alternatively one inhCCP can be fused to the amino-terminus and the same or a different one inhCCP fused to the carboxy-terminus. Alternatively, the inhCCP s and targeting ligands to either Tumor Necrosis Factor Receptors, HER2/NEU, Vascular Endothelial Growth Factor Receptors, Epithelial Growth Factor Receptors, PD-L1, PD-L2 or C1q/TNF family members can be attached to a different subunit of the scaffold oligomer than the inhCCP.

Leader sequences can be added to the amino-terminus of inhCCP and actCCP to enhance secretion.

Linker sequences can be inserted between CCP, scaffold domains and targeting domains facilitate interaction with targeted receptors and convertases and iC3b.

General Description of Fusion Protein Sequences:

The fusion proteins as described herein comprise three or four parts joined by flexible linkers (for example, those comprised of glycine, serine or alanine residues) with the number and nature of each varied by application:

A signal sequence directing export to the cell surface membrane

The CCP sequence

A scaffold sequence

Optionally a receptor targeting sequence

### Example 1: actCCP with a Signal Sequence, an Amino-Terminus CR1-Site 1 CCP with a D109N Mutation, and a C4BP Oligomerization Domain (FIG. 6)

(SEQ ID NO: 2)
MGASSPRSPEPVGPPAPGLPFCCGGSLLAVVVLIALPVAWGQCNAPEW
LPFARPTNLTDEFEFPIGTYLNYECRPGYSGRPFSIICLKNSVWTGAKD
RCRRKSCRNPPDPVNGMVHVIKGIQFGSQIKYSCTKGYRLIGSSSATCI
ISGNTVIWDNETPICDRIPCGLPPTITNGDFISTNRENFHYGSVNTTYR
CNPGSGGRKVFELVGEPSIYCTSNDDQVGIWSGPAPQCIIPNSGGGSGG
GTPEGCEQVLTGKRLMQCLPNPEDVKMALEVYKLSLEIEQLELQRDSAR
QSTLDKEL

### Example 2: actCCP with a Signal Sequence, an Amino-Terminus CR1-Site 3 CCP and a C4BP Oligomerization Domain (FIG. 6)

(SEQ ID NO: 3)
MGASSPRSPEPVGPPAPGLPFCCGGSLLAVVVLLALPVAWGHCQAPDH
FLFAKLKTQTNASDFPIGTSLKYECRPEYYGRPFSITCLDNLVWSSPKD
VCKRKSCKTPPDPVNGMVHVITDIQVGSRINYSCTTGHRLIGHSSAECI
LSGNTAHWSTKPPICQRIPCGLPPTIANGDFISTNRENFHYGSVVTYRC
NLGSRGRKVFELNGEPSIYCTSNDDQVGIWSGPAPQCIIPNSGGGSGGG
TPEGCEQVLTGKRLMQCLPNPEDVKMALEVYKLSLEIEQLELQRDSARQ
STLDKEL

### Example 3: A Signal Sequence with C4BP Oligomer Domain and a Carboxy-Terminal Single Chain C1q Fusion (FIGS. 6 and 8)

(SEQ ID NO: 4)
MWWRLWWLLLLLLLLWPMVWAAATPEGCEQVLTGKRLMQCLPNPE
DVKMALEVYKLSLEIEQLELQRDSARQSTLDKELGGGSKDQPRPAFSAI
RRNPPMGGNVVIFDTVITNQEEPYQNHSGREVCTVPGYYYFTFQVLSQW
EICLSIVSSSRGQVRRSLGFCDTTNKGLFQVVSGGMVLQLQQGDQVWVE
KDPKKGHIYQGSEADSVFSGFLIFPSAGSGKQKFQSVFTVTRQTHQPPA
PNSLIRFNAVLTNPQGDYDTSTGKFTCKVPGLYYFYHASHTANLCVLLY
RSGVKVVTFCGHTSKTNQVNSGGVLLRLQVGEEVWLAVNDYYDMVGIQG
SDSVFSGFLLFPDGSAKATQKIAFSATRTINVPLRRDQTIRFDHVITNM
NNNYEPRSGKFTCKVPGLYYFTYHASSRGNLCVNLMRGRERAQKVVTFC
DYAYNTFQVTTGGMNTLKLEQGENVFLQATDKNSLLGMEGANSIFSGFL
LFPDMEA

### Example 4: Coexpression of Proteins Derived from Example 1 or 2 with Example 3 Produces an Oligomer Mixture with Both Amino-Terminus CCP Domains and Carboxy Terminal C1q Globular Targeting Domains (FIGS. 6 and 8)

### Example 5: A Signal Sequence, an inhCCP with an Amino-Terminus VSIG4 Extracellular Domain (Residues 18-137) and a C4BP Oligomer Domain Fusion (FIG. 6)

(SEQ ID NO: 5)
MWWRLWWLLLLLLLLWPMVWAAAGRPILEVPESVTGPWKGDVNLPC
TYDPLQGYTQVLVKWLVQRGSDPVTIFLRDSSGDHIQQAKYQGRLHVSH
KVPGDVSLQLSTLEMDDRSHYTCEVTWQTPDGNQVVRDKITELRVQKSG
GGSGGGTPEGCEQVLTGKRLMQCLPNPEDVKMALEVYKLSLEIEQLELQ
RDSARQSTLDKEL

### Example 6 a Signal Sequence with C4BP Oligomer Domain and a PD1 Extracellular Domain (Residues 34-150) Fusion (FIGS. 6 and 7)

(SEQ ID NO: 6)
MWWRLWWLLLLLLLLWPMVWAAATPEGCEQVLTGKRLMQCLPNPE
DVKMALEVYKLSLEIEQLELQRDSARQSTLDKELGGGSSLTFYPAWLTV
SEGANATFTCSLSNWSEDLMLNWNRLSPSNQTEKQAAFCNGLSQPVQDA
RFQIIQLPNRHDFHMNILDTRRNDSGIYLCGAISLHPKAKIEESPGAEL
VVTERILE

### Example 7 CR1(Site 6) a Signal Sequence, CR1 Site 3, a C4BP Oligomerization Domain Fused to a PD1 Fusion Extracellular Domain (Residues 34-150) Figure (6 and 7)

(SEQ ID NO: 7)
MWWRLWWLLLLLLLLWPMVWAAAGHCQAPDHFLFAKLKTQTNASD
FPIGTSLKYECRPEYYGRPFSITCLDNLVWSSPKDVCKRKSCKTPPDPV
NGMVHVITDIQVGSRINYSCTTGHRLIGHSSAECILSGNTAHWSTKPPI
CQRIPCGLPPTIANGDFISTNRENFHYGSVVTYRCNLGSRGRKVFELVG
EPSIYCTSNDDQVGIWSGPAPQCIIPNSGGGGSGGGGSGGGGSTPEGCE
QVLTGKRLMQCLPNPEDVKMALEVYKISLEIEQLELQRDSARQSTLDKE
LSGGGSGGGSLTFYPAWLTVSEGANATFTCSLSNWSEDLMLNWNRLSPS
NQTEKQAAFCNGLSQPVQDARFQIIQLPNRHDFHMNILDTRRNDSGIYL
CGAISLHPKAKIEESPGAELVVTERILE

### Example 8 a Signal Sequence, an actCR1 Site 3, a High Affinity Binding PD-1 Mutant [6, 7] and an actCR1 Site 1 D109N Mutant Fusion (FIG. 7)

(SEQ ID NO: 8
MWWRLWWLLLLLLLLWPMVWAAAGHCQAPDHFLFAKLKTQTNASD
FPIGTSLKYECRPEYYGRPFSITCLDNLVWSSPKDVCKRKSCKTPPDPV
NGMVHVITDIQVGSRINYSCTTGHRLIGHSSAECILSGNTAHWSTKPPI
CQRIPCGLPPTIANGDFISTNRENFHYGSVVTYRCNLGSRGRKVFELVG
EPSIYCTSNDDQVGIWSGPAPQCIIPNSGGGGSGGGGSGGGGSDSPDRP
WNPPTFSPALLVVTEGDNATFTCSFSNTSESFHVIWHRESPSGQTDTLA
AFPEDRSQPGQDCRFRVTQLPNCRDFHMSVVRARRNDSGTYVCGVISLA
PKIQIKESLRAELRVTERRSGGGGSGGGGSGGGGSGQCNAPEWLPFARP
TNLTDEFEFPIGTYLNYECRPGYSGRPFSIICLKNSVWTGAKDRCRRKS
CRNPPDPVNGMVHVIKGIQFGSQIKYSCTKGYRLTGSSSATCIISGNTV
IWDNETPICDRIPCGLPPTITNGDFISTNRENFHYGSVVTYRCNPGSGG
RKVFELVGEPSIYCTSNDDQVGIWSGPAPQCIIPN

### Example 9

Constructs described herein can also comprise an antigen targeting domain producing antigen-specific constructs for ERBB2, CEACAM5, CD47, EGFR and CD274 as shown in Table 4 and sequences shown in FIG. 10.

TABLE 4
Antigen-Specific Constructs. The target and the crystal
structure accession number are given (www.rcsb.org).
CEACAM5 is referred to as CEA and CD274 as PD-L1

| Target | Structure |
|---|---|
| ERBB2 | 3H3B |
| CEACAM5 | 1QOK |
| CD47 | 5IWL |
| EGFR | 4UIP |
| CD274 | 5JDS |

### LIST OF REFERENCES

The references described herein are incorporated by reference in their entirety.
1. Elvington, M., Liszewski, M. K. & Atkinson, J. P. Evolution of the complement system: from defense of the single cell to guardian of the intravascular space. Immunological reviews 274, 9-15 (2016).
2. Hajishengallis, G., Reis, E. S., Mastellos, D. C., Ricklin, D. & Lambris, J. D. Novel mechanisms and functions of complement. Nature immunology 18, 1288-1298 (2017).
3. Nonaka, M. Evolution of the complement system. Sub-cellular biochemistry 80, 31-43 (2014).4. Law, S. K. & Dodds, A. W. The internal thioester and the covalent binding properties of the complement proteins C3 and C4. Protein science: a publication of the Protein Society 6, 263-74 (1997).
4. Law, S. K. & Dodds, A. W. The internal thioester and the covalent binding properties of the complement proteins C3 and C4. Protein science: a publication of the Protein Society 6, 263-74 (1997).
5. Makou, E., Herbert, A. P. & Barlow, P. N. Creating functional sophistication from simple protein building blocks, exemplified by factor H and the regulators of complement activation. Biochemical Society transactions 43, 812-8 (2015).
6. Kirkitadze, M. D. & Barlow, P. N. Structure and flexibility of the multiple domain proteins that regulate complement activation. Immunological reviews 180, 146-61 (2001).
7. Li, M. F., Sui, Z. H. & Sun, L. A teleost CD46 is involved in the regulation of complement activation and pathogen infection. Scientific reports 7, 15028 (2017).
8. Krych-Goldberg, M. & Atkinson, J. P. Structure-function relationships of complement receptor type 1. Immunological reviews 180, 112-22 (2001).
9. Schmidt, C. Q., Lambris, J. D. & Ricklin, D. Protection of host cells by complement regulators. Immunological reviews 274, 152-171 (2016).
10. Forneris, F., Wu, J., Xue, X., Ricklin, D., Lin, Z., Sfyroera, G. et al. Regulators of complement activity mediate inhibitory mechanisms through a common C3b-binding mode. The EMBO journal 35, 1133-49 (2016).
11. Birmingham, D. J., Chen, W., Liang, G., Schmitt, H. C., Gavit, K. & Nagaraja, H. N. A CR1 polymorphism associated with constitutive erythrocyte CR1 levels affects binding to C4b but not C3b. Immunology 108, 531-8 (2003).
12. Shichishima, T. Glycosylphosphatidylinositol (GPI)-anchored membrane proteins in clinical pathophysiology of paroxysmal nocturnal hemoglobinuria (PNH). Fukushima journal of medical science 41, 1-13 (1995).
13. Hofmeyer, T., Schmelz, S., Degiacomi, M. T., Dal Peraro, M., Daneschdar, M., Scrima, A. et al. Arranged sevenfold: structural insights into the C-terminal oligomerization domain of human C4b-binding protein. Journal of molecular biology 425, 1302-17 (2013).
14. Nakao, M. & Somamoto, T. The Evolution of Complement System Functions and Pathways in Vertebrates. in The evolution of the immune system: conservation and diversification (ed. Malagoli, D.) 151-171 (Elsevier, Amsterdam, 2016).
15. Ricklin, D., Mastellos, D. C., Reis, E. S. & Lambris, J. D. The renaissance of complement therapeutics. Nature reviews. Nephrology 14, 26-47 (2018).
16. Harder, M. J., Anliker, M., Hochsmann, B., Simmet, T., Huber-Lang, M., Schrezenmeier, H. et al. Comparative Analysis of Novel Complement-Targeted Inhibitors, MiniFH, and the Natural Regulators Factor H and Factor H-like Protein 1 Reveal Functional Determinants of Complement Regulation. Journal of immunology 196, 866-76 (2016).
17. Schmidt, C. Q., Bai, H., Lin, Z., Risitano, A. M., Barlow, P. N., Ricklin, D. et al. Rational engineering of a minimized immune inhibitor with unique triple-targeting properties. Journal of immunology 190, 5712-21 (2013).
18. Fridkis-Hareli, M., Storek, M., Mazsaroff, I., Risitano, A. M., Lundberg, A. S., Horvath, C. J. et al. Design and development of TT30, a novel C3d-targeted C3/C5 convertase inhibitor for treatment of human complement alternative pathway-mediated diseases. Blood 118, 4705-13 (2011).
19. Chen, Q., Manzke, M., Hartmann, A., Buttner, M., Amann, K., Pauly, D. et al. Complement Factor H-Related 5-Hybrid Proteins Anchor Properdin and Activate Complement at Self-Surfaces. Journal of the American Society of Nephrology: JASN 27, 1413-25 (2016).
20. Togarsimalemath, S. K., Sethi, S. K., Duggal, R., Le Quintrec, M., Jha, P., Daniel, R. et al. A novel CFHR1-CFHR5 hybrid leads to a familial dominant C3 glomerulopathy. Kidney international 92, 876-887 (2017).
21. Hu, C., Li, L., Ding, P., Ge, X., Zheng, L., Wang, X. et al. Complement Inhibitor CRIg/FH Ameliorates Renal Ischemia Reperfusion Injury via Activation of PI3K/AKT Signaling. Journal of immunology 201, 3717-3730 (2018).
22. Brune, K. D., Buldun, C. M., Li, Y., Taylor, I. J., Brod, F., Biswas, S. et al. Dual Plug-and-Display Synthetic Assembly Using Orthogonal Reactive Proteins for Twin Antigen Immunization. Bioconjugate chemistry 28, 1544-1551 (2017).
23. Ogun, S. A., Dumon-Seignovert, L., Marchand, J. B., Holder, A. A. & Hill, F. The oligomerization domain of C4-binding protein (C4 bp) acts as an adjuvant, and the fusion protein comprised of the 19-kilodalton merozoite surface protein 1 fused with the murine C4 bp domain protects mice against malaria. Infection and immunity 76, 3817-23 (2008).
24. Valldorf, B., Fittler, H., Deweid, L., Ebenig, A., Dickgiesser, S., Sellmann, C. et al. An Apoptosis-Inducing Peptidic Heptad That Efficiently Clusters Death Receptor 5. Angewandte Chemie 55, 5085-9 (2016).
25. Oudin, S., Libyh, M. T., Goossens, D., Dervillez, X., Philbert, F., Reveil, B. et al. A soluble recombinant multimeric anti-Rh(D) single-chain Fv/CR1 molecule restores the immune complex binding ability of CR1-deficient erythrocytes. Journal of immunology 164, 1505-13 (2000).
26. Shapiro, L. & Scherer, P. E. The crystal structure of a complement-1q family protein suggests an evolutionary link to tumor necrosis factor. Current biology: CB 8, 335-8 (1998).
27. Zipfel, P. F. & Skerka, C. Complement regulators and inhibitory proteins. Nature reviews. Immunology 9, 729-40 (2009).
28. Goicoechea de Jorge, E., Caesar, J. J., Malik, T. H., Patel, M., Colledge, M., Johnson, S. et al. Dimerization of complement factor H-related proteins modulates complement activation in vivo. Proceedings of the National Academy of Sciences of the United States of America 110, 4685-90 (2013).
29. Skerka, C., Chen, Q., Fremeaux-Bacchi, V. & Roumenina, L. T. Complement factor H related proteins (CFHRs). Molecular immunology 56, 170-80 (2013).
30. Wiesmann, C., Katschke, K. J., Yin, J., Helmy, K. Y., Steffek, M., Fairbrother, W. J. et al. Structure of C3b in complex with CRIg gives insights into regulation of complement activation. Nature 444, 217-20 (2006).
31. Clark, S. J. & Bishop, P. N. Role of Factor H and Related Proteins in Regulating Complement Activation in the Macula, and Relevance to Age-Related Macular Degeneration. Journal of clinical medicine 4, 18-31 (2015).
32. Ressl, S., Vu, B. K., Vivona, S., Martinelli, D. C., Sudhof, T. C. & Brunger, A. T. Structures of C1q-like proteins reveal unique features among the C1q/TNF superfamily. Structure 23, 688-99 (2015).
33. Maass, F., Wustehube-Lausch, J., Dickgiesser, S., Valldorf, B., Reinwarth, M., Schmoldt, H. U. et al. Cystine-knot peptides targeting cancer-relevant human cytotoxic T lymphocyte-associated antigen 4 (CTLA-4). Journal of peptide science: an official publication of the European Peptide Society 21, 651-60 (2015).
34. Moreau, C., Bally, I., Chouquet, A., Bottazzi, B., Ghebrehiwet, B., Gaboriaud, C. et al. Structural and Functional Characterization of a Single-Chain Form of the Recognition Domain of Complement Protein C1q. Frontiers in immunology 7, 79 (2016).
35. Strohl, W. R. Current progress in innovative engineered antibodies. Protein & cell 9, 86-120 (2018).36. ES2534675T3 “Polypeptide crig for the prevention and treatment of disorders associated complement” Genentech
36. ES2534675T3 “Polypeptide crig for the prevention and treatment of disorders associated complement” Genentech
37. WO2006042329A8 “Crig polypeptide for prevention and treatment of complement-associated disorders”
38. US20080311106A1 Product Comprising a C4 bp Core Protein and a monomeric Antigen, and Its Use
39. WO2005051414A1
40. EP1963361B1
41. U.S. Pat. No. 6,521,450B1
42. Zhou, H., et al. (2011) Structural Insights into the Down-regulation of Overexpressed pI85her2/neuProtein of Transformed Cells by the Antibody chA21. Journal of Biological Chemistry 286, 31676-31683
43. Boehm, M. K., et al. (2000) Crystal structure of the anti-(carcinoembryonic antigen) single-chain Fv antibody MFE-23 and a model for antigen binding based on intermolecular contacts. Biochem J 346 Pt 2, 519-528
44. Weiskopf, K., et al. (2016) CD47-blocking immunotherapies stimulate macrophage-mediated destruction of small-cell lung cancer. Journal of Clinical Investigation 126, 2610-2620
45. Lee, J.-j., et al. (2015) Enzymatic Prenylation and Oxime Ligation for the Synthesis of Stable and Homogeneous Protein-Drug Conjugates for Targeted Therapy. Angewandte Chemie International Edition 54, 12020-12024
46. Zhang, F., et al. (2017) Structural basis of a novel PD-L1 nanobody for immune checkpoint blockade. Cell Discovery 3, 17004
47. Alphey, M. S., et al. (2003) High Resolution Crystal Structures of Siglec-7. Journal of Biological Chemistry 278, 3372-3377

While this invention has been particularly shown and described with references to preferred embodiments thereof, it will be understood by those skilled in the art that various changes in form and details may be made therein without departing from the scope of the invention encompassed by the appended claims.

1-24. (canceled)

25. A nucleic acid construct comprising:
a.) one or more Complement Control Protein (CCP) domain sequences, wherein the CCP domain is an activating CCP domain (actCCP), or an inhibitory CCP domain (inhCCP);
b.) a scaffold protein domain sequence;
c.) a targeting domain sequence; and optionally
d.) a signal domain sequence,
wherein the sequences of a.)-d.) above are joined with flexible linkers to form a construct.

26. The activating CCP domain of claim 25, wherein the activating CCP domain is a domain derived from the group consisting of: C3d or C3dg, or a biologically active variant thereof.

27. The inhibitory CCP domain of claim 25, wherein the inhibitory CCP domain is a domain derived from the group consisting of: iC3b or VSIG4, or a biologically active variant thereof.

28. The construct of claim 25, wherein the scaffold protein domain sequence comprises all, or a biologically active portion of a sequence selected from the group consisting of: C4BPα oligomer domain, the C4BPβ oligomer domain, Tumor Necrosis Factor Receptors, HER2/NEU, Vascular Endothelial Growth Factor Receptors, Epithelial Growth Factor Receptors, PD-L1, PD-L2 or C1q/TNF family members.

29. The construct of claim 25, wherein the same activating or inhibitory CCP domain sequence is linked to both the amino and carboxy terminus of the scaffold domain sequence.

30. The construct of claim 25, wherein the activating or inhibitory CCP domain sequence is linked to one end of the scaffold sequence and a targeting sequence is linked to the other end of the scaffold.

31. The construct of claim 25, wherein the construct comprises SEQ ID NO: 5.

32. The construct of claim 25, wherein the targeting domain sequence is a ligand binding domain or receptor on a cell.

33. A method of enhancing the immunogenicity of an antigen by using actCCP, the method comprising contacting a cell with the construct of claim 25, wherein the activating CCP domain is a domain derived from the group consisting of: C3d or C3dg, or a biologically active variant thereof and wherein the construct is expressed in the cell resulting in the increased the expression of C3d or C3dg, or biologically active variants thereof including peptides derived from C3 and C4 in the cell or the cell microenvironment.

34. A method of decreasing the immunogenicity of an antigen by using inhCCP, the method comprising contacting a cell with the construct of claim 25, wherein the inhibitory CCP domain is a domain derived from the group consisting of: iC3b or VSIG4, or a biologically active variant thereof and wherein the construct is expressed in the cell resulting in increased the expression of iC3b or VSIG4, or a biologically active variant thereof including peptides derived from C3 in the cell or the cell microenvironment.

35. The method of claim 33, wherein the construct is targeted for delivery to surface of a cell using a viral vector, nanoparticle, liposome or exosome.

36. The method of claim 34, wherein the construct is targeted for delivery to surface of a cell using a viral vector, nanoparticle, liposome or exosome.

37. The method of claim 33, additionally comprising a second construct, wherein the second construct comprises an expression vector that increases expression of an antigen to induce an immune response specific for that antigen whereas both the actCCP and antigen are expressed on the surface of the same cell, or to inhibit an immune response whereas both the inhCCP and antigen are expressed on the surface of the same cell.

38. The method of claim 34, additionally comprising a second construct, wherein the second construct comprises an expression vector that increases expression of an antigen to induce an immune response specific for that antigen whereas both the actCCP and antigen are expressed on the surface of the same cell, or to inhibit an immune response whereas both the inhCCP and antigen are expressed on the surface of the same cell.

39. The method of claim 33, wherein the antigen is tagged by GPI to co-localize it to the same site on the surface of the cell as the activating or inhibitory CCP domain construct.

40. The method of claim 34, wherein the antigen is tagged by GPI to co-localize it to the same site on the surface of the cell as the activating or inhibitory CCP domain construct.

41. A method of treating cancer, or preventing metastasis of cancer, the method comprising administering to the subject a therapeutically effective amount of the activating CCP domain construct of claim 25, wherein the activating CCP domain is a domain derived from the group consisting of: C3d or C3dg, or a biologically active variant thereof and wherein expression of the construct increases localization of actCCPs to cancer cells or other cells that present tumor antigens to the immune system, such as dendritic cells, thereby enhancing the immunogenicity of the cancer cells and tumor antigens and treating the cancer.

42. The method of claim 41, wherein a therapeutically effective amount of a second construct is administered with the first construct, wherein the second construct increases the expression of a tumor-expressed antigen in cells or the micro-environment, thereby treating cancer, or preventing metastasis of cancer, or protecting against a reoccurrence of cancer in the subject by inducing an immune response to tumor-specific antigens.

43. A method of treating inflammatory disease, the method comprising administering to the subject a therapeutically effective amount of the construct of claim 25, wherein the inhibitory CCP domain is a domain derived from the group consisting of: iC3b or VSIG4, or a biologically active variant thereof and wherein expression of the construct increases localization of inhCCPs to cells that present antigens to the immune system, such as dendritic cells, thereby decreasing the immune response and ameliorating the inflammation.

44. The method of claim 43, wherein a therapeutically effective amount of a second reagent is administered with the first construct, wherein the second construct reagent also decreases inflammatory responses.
